(19) United States
(12) Patent Application Publication
(10) Pub. No.: US 2008/0058293 A1
(43) Pub. Date: Mar. 6, 2008

(54) Quinoline Derivates and Their Use in Therapy

(76) Inventors: Rhonan Ford, Leicestershire (GB); Toby Thompson, Leicestershire (GB); Paul Willis, Leicestershire (GB)

Correspondence Address: FISH & RICHARDSON P.C., P.O BOX 1022, MINNEAPOLIS, MN, 55440-1022, US

(21) Appl. No.: 10/566,320

(22) PCT Filed: Jul. 21, 2004
(86) PCT No.: PCT/SE04/01144

§ 371 Date: Jan. 26, 2006

(30) Foreign Application Priority Data
Jul. 28, 2003 (SE) 0302139-1

(51) Int. Cl. A61K 31/47; A61K 31/4709; A61K 31/497; A61P 11/00; A61P 29/00; A61P 9/10; C07D 215/38; C07D 215/48; C07D 403/04

(52) U.S. Cl. 514/162; 514/253.06; 514/311; 514/313; 544/363; 546/171

(57) ABSTRACT

The invention provides compounds of formula (I) wherein n, p, q, X, R¹, R², R³, R⁴, R⁵ and R⁶ are as defined in the specification; processes for their preparation, pharmaceutical compositions containing them and their use in therapy.

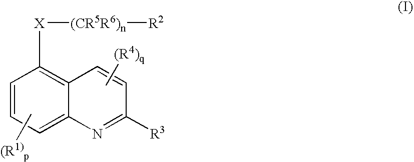

The present invention relates to certain heteroaryl amide derivatives, processes for their preparation, pharmaceutical compositions containing them, and their use in therapy.

The P2X₇ receptor (previously known as P2Z receptor), which is a ligand-gated ion channel; is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells), apoptosis and L-selectin shedding (lymphocytes). P2X₇ receptors are also located on antigen-presenting cells (APC), keratinocytes, salivary acinar cells (parotid cells), hepatocytes and mesangial cells.

It would be desirable to make compounds effective as P2X₇ receptor antagonists for use in the treatment of inflammatory, immune or cardiovascular diseases, in the aetiologies of which the P2X₇ receptor may play a role.

The present invention provides a compound of formula

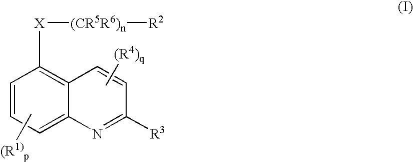

or a pharmaceutically acceptable salt or solvate thereof, wherein

p is 0, 1 or 2;

each R¹ independently represents halogen or C₁-C₆ alkyl optionally substituted by at least one substituent selected from hydroxyl, halogen and C₁-C₆ alkoxy;

X is C(O)NH or NHC(O);

n is 1, 2, 3, 4 or 5;

within each grouping, CR⁵R⁶, R⁵ and R⁶ each independently represent hydrogen, halogen, phenyl or C₁-C₆ alkyl, or R⁵ and R⁶ together with the carbon atom to which they are both attached form a C₃-C₈ cycloalkyl ring;

R² represents an unsaturated 4- to 10-membered ring system which may comprise at least one ring heteroatom selected from nitrogen, oxygen and sulphur, the ring system being optionally substituted with at least one substituent selected from halogen, —COOR¹³, hydroxyl, —NR¹⁴R¹⁵, —CONR¹⁶R¹⁷, —SO₂NR¹⁸R¹⁹, —NR²⁰SO₂R²¹, C₁-C₆ alkyl, C₁-C₆ alkylcarbonyl, C₁-C₆ alkoxy, C₁-C₆ alkylcarbonyloxy, C₁-C₆ alkoxycarbonyl, C₁-C₆ hydroxyalkyl and —S(O)ₘC₁-C₆ alkyl where m is 0, 1 or 2;

R³ represents hydrogen or a group —R⁷, —OR⁷, —SR⁷ or —NR⁷R⁸;

q is 0, 1 or 2;

each R⁴ independently represents halogen or C₁-C₆ alkyl optionally substituted by at least one substituent selected from hydroxyl, halogen and C₁-C₆ alkoxy;

R⁷ and R⁸ each independently represent hydrogen, C₁-C₁₀ alkyl, C₃-C₈ cycloalkyl or a saturated or unsaturated 3- to 10-membered heterocyclic ring system comprising at least one ring heteroatom selected from nitrogen, oxygen and sulphur, the alkyl, cycloalkyl and heterocyclic ring system each being optionally substituted with at least one substituent selected from halogen, hydroxyl, C₁-C₆ alkoxy, C₁-C₆ alkylthio, C₁-C₆ hydroxyalkyl, C₁-C₆ hydroxyalkoxy, C₁-C₆ alkoxycarbonyl, C₃-C₈ cycloalkyl, —NR⁹R¹⁰, —COOR²², —CONR²³R²⁴, —SO₂NR²⁵R²⁶, —NR²⁷SO₂R²⁸ and ZR⁶⁸ or

alternatively, R⁷ and R⁸ may together with the nitrogen atom to which they are attached form a 4- to 7-membered saturated heterocyclic ring that optionally further comprises one or two ring heteroatoms independently selected from nitrogen, oxygen and sulphur and that optionally further comprises a bridging group, the heterocyclic ring being optionally substituted with at least one substituent selected from halogen, hydroxyl, cyano, C₁-C₆ alkyl, C₁-C₆ alkoxy, C₁-C₆ alkylthio, C₁-C₆ hydroxyalkyl, C₁-C₆ hydroxyalkoxy, C₁-C₆ alkoxycarbonyl, C₃-C₈ cycloalkyl, —NR¹¹R¹², —COOR²⁹, —CONR³⁰R³¹, —SO₂NR³²R³³, —NR³⁴SO₂R³⁵, Z′R⁶⁹, (CH₂)₁₋₆NR⁷⁰R⁷¹, SO₂R⁷², NR⁷³CONR⁷⁴SO₂R⁷⁵ or M(CH₂)₁₋₆COOR⁷⁶ wherein M represents a bond, O, S, SO, SO₂, and a group >NR⁷⁷;

R⁹ and R¹⁰ each independently represent hydrogen or a C₁-C₆ alkylcarbonyl, C₂-C₇ alkenyl or C₁-C₇ alkyl group, each group being optionally substituted with at least one substituent selected from hydroxyl, —NR³⁶R³⁷, —COOR³⁸, —CONR³⁹R⁴⁰, —SO₂NR⁴¹R⁴², —NR⁴³SO₂R⁴⁴, C₁-C₆ alkoxy, C₁-C₆ alkylthio, C₁-C₆ alkoxycarbonyl and a saturated or unsaturated 3- to 10-membered ring system which may comprise at least one ring heteroatom selected from nitrogen, oxygen and sulphur, the ring system in turn being optionally substituted with at least one substituent selected from halogen, hydroxyl, oxo, carboxyl, cyano, C₁-C₆ alkyl and C₁-C₆ hydroxyalkyl, or

alternatively, R⁹ and R¹⁰ may together with the nitrogen atom to which they are attached form a 4- to 7-membered saturated heterocyclic ring that optionally further comprises one or two ring heteroatoms independently selected from nitrogen, oxygen and sulphur, the heterocyclic ring being optionally substituted with at least one substituent selected from —OR⁵⁴, —NR⁵⁵R⁵⁶, —(CH₂)ₜ—NR⁵⁷R⁵⁸ where t is 1, 2, 3, 4, 5 or 6, —COOR⁵⁹, —CONR⁶⁰R⁶¹, —SO₂NR⁶²R⁶³, —NR⁶⁴SO₂R⁶⁵, C₁-C₆ hydroxyalkyl, C₁-C₆ alkoxy, C₁-C₆ alkylthio, C₁-C₆ alkoxycarbonyl and Z″R⁸⁰;

R¹¹ and R¹² each independently represent hydrogen or a C₁-C₆ alkylcarbonyl, C₁-C₆ alkoxycarbonyl, C₂-C₇ alkenyl or C₁-C₇ alkyl group, each group being optionally substituted with at least one substituent selected from hydroxyl, —NR⁴⁵R⁴⁶, —COOR⁴⁷, —CONR⁴⁸R⁴⁹, —SO₂NR⁵⁰R⁵¹, —NR⁵²SO₂R⁵³, —NR⁶⁶C(O)R⁶⁷, C₁-C₆ alkoxy, C₁-C₆ alkylthio and C₁-C₆ alkoxycarbonyl;

Z, Z′ and Z″ independently represent a bond, O, S, SO, SO₂, >NR⁷⁸, C₁-₆ alkylene, or a group —O(CH₂)₁₋₆—, —NR⁷⁹(CH₂)₁₋₆— or —S(O)p(CH₂)₁₋₆— wherein p is 0, 1 or 2;

R⁶⁸, R⁶⁹ and R⁸⁰ independently represent tetrazolyl or a 5- to 6-membered heterocyclic ring comprising from 1 to 4 heteroatoms selected from nitrogen, oxygen and sulphur, which heterocyclic ring is substituted by at least one substituent selected from hydroxyl, ═O, and ═S, and which heterocyclic ring may further be optionally substituted by at least one substituent selected from halogen , nitro, cyano, —SO₂C₁₋₆ alkyl, C₁₋₆ alkoxycarbonyl, and a C₁₋₆ alkyl group which C₁₋₆ alkyl group can be optionally substituted by at least one substituent selected from halogen and hydroxyl;

R¹³, R¹⁴, R¹⁵, R¹⁶, R¹⁷, R¹⁸, R¹⁹, R²⁰ and R²¹ each independently represent hydrogen or C₁-C₆ alkyl optionally substituted by at least one substituent selected from hydroxyl, halogen and C₁-C₆ alkoxy;

R²², R²³, R²⁴, R²⁵, R²⁶, R^{27 , R}²⁸, R²⁹, R³⁰, R³¹, R³², R³³, R³⁴ and R³⁵ each independently represent hydrogen or C₁-C₆ alkyl optionally substituted by at least one substituent selected from hydroxyl, halogen and C₁-C₆ alkoxy;

R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵² and R⁵³ each independently represent hydrogen or C₁-C₆ alkyl optionally substituted by at least one substituent selected from hydroxyl, halogen and C₁-C₆ alkoxy;

R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶ and R⁶⁷ each independently represent hydrogen or C₁-C₆ alkyl optionally substituted by at least one substituent selected from hydroxyl, halogen and C₁-C₆ alkoxy; and

R⁷⁰, R⁷¹, R⁷², R⁷³, R⁷⁴, R⁷⁵, R⁷⁶, R⁷⁷, R⁷⁸ and R⁷⁹ each independently represent hydrogen or C₁-C₆ alkyl optionally substituted by at least one substituent selected from hydroxyl, halogen and C₁-C₆ alkoxy;

with the provisos that:
(a) when X represents NHC(O), p is 0, q is 0, n is 1 and R³, R⁵ and R⁶ each independently represent hydrogen, then R² is other than a 2-carboxy-phenyl group; and
(b) when X represents NHC(O), p is 0, q is 0, n is 2, R³ represents hydrogen and each R⁵ and R⁶ independently represents hydrogen, then R² is other than a 3,4-diamino-phenyl group or a 5-methyl-2-furanyl group; and
(c) when X represents C(O)NH, p is 0, q is 0, n is 2, R³ represents hydrogen and each R⁵ and R⁶ independently represents hydrogen, then R² is other than an unsubstituted phenyl group, an unsubstituted 1H-indol-3-yl group, or a 2-methyl-1H-indol-3-yl group.

In the context of the present specification, unless otherwise indicated, an alkyl or alkenyl substituent or an alkyl or alkenyl moiety in a substituent group may be linear or branched. Examples of alkyl groups/moieties containing up to 7 carbon atoms include methyl, ethyl, n-propyl, isopropyl, n-butyl, isobutyl, tert-butyl, n-pentyl, n-hexyl and n-heptyl. A hydroxyalkyl or hydroxyalkoxy substituent may contain one or more hydroxyl groups but preferably contains one or two hydroxyl groups. When R⁷ and R⁸ (or R⁹ and R¹⁰) represent a 4- to 7-membered saturated heterocycle, it should be understood that the heterocycle will contain no more than three ring heteroatoms: the nitrogen ring atom to which R⁷ and R⁸ (or R⁹ and R¹⁰) are attached and optionally one or two further ring heteroatoms independently selected from nitrogen, oxygen and sulphur. When either of R⁷ and R⁸ represents a saturated or unsaturated 3- to 10-membered heterocyclic ring system, it should be understood that the ring system may have alicyclic or aromatic properties. Furthermore, an unsaturated ring system will be partially or fully unsaturated. The same comments apply to the saturated or unsaturated 3- to 10-membered ring system in the definition of R⁹/R¹⁰. Similarly, the unsaturated 4- to 10-membered ring system in the definition of R² may be fully or partially unsaturated.

Each R¹ independently represents halogen (e.g. chlorine, fluorine, bromine or iodine), or C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one substituent (e.g. one, two or three substituents independently) selected from hydroxyl, halogen (e.g. chlorine, fluorine, bromine or iodine) and C₁-C₆, preferably C₁-C₄, alkoxy (e.g. methoxy, ethoxy, n-propoxy or n-butoxy).

In an embodiment of the invention, p is 0 or p is 1 and R¹ represents halogen, in particular chlorine.

In an embodiment of the invention, n is 1, 2, 3 or 4. In another embodiment, n is 1, 2 or 3. In yet another embodiment, n is 2.

Within each grouping, CR⁵R⁶, R⁵ and R⁶ each independently represent hydrogen, halogen (e.g. chlorine, fluorine, bromine or iodine), phenyl or C₁-C₆, preferably C₁-C₄, alkyl (e.g. methyl, ethyl, n-propyl, isopropyl, n-butyl, isobutyl, tert-butyl, n-pentyl or n-hexyl), or R⁵ and R⁶ together with the carbon atom to which they are both attached form a C₃-C₈, preferably C₅-C₆, cycloalkyl ring (e.g. cyclopropyl, cyclobutyl, cyclopentyl or cyclohexyl).

In an embodiment of the invention, R⁵ and R⁶ each independently represent hydrogen, halogen, or C₁-C₆ alkyl, or R⁵ and R⁶ together with the carbon atom to which they are both attached form a C₃-C₈ cycloalkyl ring.

In another embodiment of the invention, R⁵ and R⁶ each independently represent hydrogen or C₁-C₄ alkyl, in particular methyl.

R² represents an unsaturated 4- to 10-membered, preferably 4- to 9-membered, more preferably 4- to 6-membered, ring system which may comprise at least one ring heteroatom (e.g. one, two, three or four ring heteroatoms independently) selected from nitrogen, oxygen and sulphur, the ring system being optionally substituted with at least one substituent (e.g. one, two, three or four substituents independently) selected from halogen (e.g. chlorine, fluorine, bromine or iodine), —COOR¹³, hydroxyl, —NR¹⁴R¹⁵, —CONR¹⁶R¹⁷, —SO₂NR¹⁸R¹⁹, —NR²⁰SO₂R²¹, C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, alkylcarbonyl (e.g. methylcarbonyl or ethylcarbonyl), C₁-C₆, preferably C₁-C₄, alkoxy (e.g. methoxy, ethoxy, n-propoxy or n-butoxy), C₁-C₆, preferably C₁-C₄, alkylcarbonyloxy (e.g. methylcarbonyloxy or ethylcarbonyloxy), C₁-C₆, preferably C₁-C₄, alkoxycarbonyl (e.g. methoxycarbonyl or ethoxycarbonyl), C₁-C₆, preferably C₁-C₄, hydroxyalkyl (e.g. —CH₂OH, —CH₂CH₂OH, —CH₂CH₂CH₂OH or —CH(OH)CH₃) and —S(O)ₘC₁-C₆, preferably C₁-C₄, alkyl where m is 0, 1 or 2 (e.g. methylthio, ethylthio, methylsulphinyl, ethylsulphinyl, methylsulphonyl or ethylsulphonyl)

In R², the unsaturated 4- to 10-membered ring system may be monocyclic or polycyclic (e.g. bicyclic) and may be partially or fully unsaturated. Examples of ring systems that may be used include one or more (in any combination) of cyclopentenyl, cyclohexenyl, phenyl, pyrazolyl, thiazolidinyl, indanyl, thienyl, isoxazolyl, thiadiazolyl, pyrrolyl, furyl, thiazolyl, indolyl, imidazolyl, benzimidazolyl, triazolyl, tetrazolyl, pyridinyl, pyrimidinyl or pyrazinyl. Preferred ring systems include phenyl, furyl, thienyl and pyridinyl.

In an embodiment of the invention, R² represents an unsaturated 4-, 5- or 6-membered ring optionally comprising one ring heteroatom selected from nitrogen, oxygen and sulphur, the ring being optionally substituted with at least one substituent (e.g. one, two, three or four substituents independently) selected from halogen, —COOR¹³, hydroxyl, —NR¹⁴R¹⁵, —CONR¹⁶R¹⁷, —SO₂NR¹⁸R¹⁹, —NR²⁰SO₂R²¹, C₁-C₄ alkyl, C₁-C₄ alkylcarbonyl, C₁-C₄ alkoxy, C₁-C₄ alkylcarbonyloxy, C₁-C₄ alkoxycarbonyl, C₁-C₄ hydroxyalkyl and —S(O)ₘC₁-C₄ alkyl where m is 0, 1 or 2.

In another embodiment of the invention, R² represents an unsaturated 6-membered ring optionally substituted with at least one substituent (e.g. one or two substituents independently) selected from halogen (particularly chlorine) and C₁-C₄ alkoxy (particularly methoxy).

Each R⁴ independently represents halogen (e.g. chlorine, fluorine, bromine or iodine), or C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one substituent (e.g. one, two or three substituents independently) selected from hydroxyl, halogen (e.g. chlorine, fluorine, bromine or iodine) and C₁-C₆, preferably C₁-C₄, alkoxy (e.g. methoxy, ethoxy, n-propoxy or n-butoxy).

In an embodiment of the invention, q is 0 or q is 1 and R⁴ represents halogen, in particular chlorine.

In an embodiment of the invention, R³ represents a group —R⁷, —OR⁷, —SR⁷ or —NR⁷R⁸.

In another embodiment of the invention, R³ represents hydrogen or a group —R⁷ or —NR⁷R⁸.

R⁷ and R⁸ each independently represent hydrogen, C₁-C₁₀, preferably C₁-C₆, alkyl (e.g. methyl, ethyl, n-propyl, isopropyl, n-butyl, isobutyl, tert-butyl, n-pentyl, n-hexyl, n-heptyl, n-octyl, n-nonyl or n-decyl), C₃-C₈, preferably C₅-C₆, cycloalkyl (e.g. cyclopropyl, cyclobutyl, cyclopentyl or cyclohexyl) or a saturated or unsaturated 3- to 10-membered heterocyclic ring system comprising at least one ring heteroatom (e.g. one, two, three or four ring heteroatoms independently) selected from nitrogen, oxygen and sulphur, the alkyl, cycloalkyl and heterocyclic ring system each being optionally substituted with at least one substituent (e.g. one, two, three or four substituents independently) selected from halogen (e.g. chlorine, fluorine, bromine or iodine), hydroxyl, C₁-C₆, preferably C₁-C₄, alkoxy (e.g. methoxy, ethoxy, n-propoxy or n-butoxy), C₁-C₆, preferably C₁-C₄, alkylthio (e.g. methylthio, ethylthio, n-propylthio or n-butylthio), C₁-C₆, preferably C₁-C₄, hydroxyalkyl (e.g. —CH₂OH, —CH₂CH₂OH, —CH₂CH₂CH₂OH or —CH(OH)CH₃), C₁-C₆, preferably C₁-C₄, hydroxyalkoxy (e.g. —O—CH₂CH₂OH or —O—CH₂CH₂CH₂OH), C₁-C₆, preferably C₁-C₄, alkoxycarbonyl (e.g. methoxycarbonyl or ethoxycarbonyl), C₃-C₈, preferably C₅-C₆, cycloalkyl (e.g. cyclopropyl, cyclobutyl, cyclopentyl or cyclohexyl), —NR⁹R¹⁰, —COOR²², —CONR²³R²⁴, —SO₂NR²⁵R²⁶, —NR²⁷SO₂R²⁸ and ZR⁶⁸.

Examples of saturated or unsaturated 3- to 10-membered heterocyclic ring systems R⁷ and R⁸, which may be monocyclic or polycyclic (e.g. bicyclic), include one or more (in any combination) of pyrrolidinyl, piperidinyl, pyrazolyl, homopiperidinyl, thiazolidinyl, thienyl, isoxazolyl, thiadiazolyl, pyrrolyl, furanyl, thiazolyl, indolyl, quinolinyl, benzimidazolyl, triazolyl, tetrazolyl and pyridinyl.

In an embodiment of the invention, R⁷ and R⁸ each independently represent hydrogen or C₁-C₁₀, preferably C₁-C₆, alkyl optionally substituted with at least one substituent (e.g. one or two substituents independently) selected from halogen, hydroxyl, C₁-C₄ alkoxy, C₁-C₄ alkylthio, C₁-C₄ hydroxyalkyl, C₁-C₄ hydroxyalkoxy, C₁-C₄ alkoxycarbonyl, C₅-C₆ cycloalkyl, —NR⁹R¹⁰, COOR²², —CONR²³R²⁴, —SO₂NR²⁵R²⁶ and —NR²⁷SO₂R²⁸.

In a further embodiment, R⁷ and R⁸ each independently represent hydrogen or C₁-C₄ alkyl optionally substituted by —NR⁹R¹⁰.

Alternatively, when R³ represents —NR⁷R⁸, R⁷ and R⁸ may together with the nitrogen atom to which they are attached form a 4- to 7-membered saturated heterocyclic ring that optionally further comprises one or two ring heteroatoms independently selected from nitrogen, oxygen and sulphur and that optionally further comprises a bridging group (e.g. pyrrolidinyl, piperidinyl, piperazinyl, morpholinyl, thiomorpholinyl or diazabicyclo[2.2.1]hept-2-yl), the heterocyclic ring being optionally substituted with at least one substituent (e.g. one, two, three or four substituents independently) selected from halogen (e.g. chlorine, fluorine, bromine or iodine), hydroxyl, C₁-C₆, preferably C₁-C₄, alkoxy (e.g. methoxy, ethoxy, n-propoxy or n-butoxy), C₁-C₆, preferably C₁-C₄, alkylthio (e.g. methylthio, ethylthio, n-propylthio or n-butylthio), C₁-C₆, preferably C₁-C₄, hydroxyalkyl (e.g. —CH₂OH, —CH₂CH₂OH, —CH₂CH₂CH₂OH or —CH(OH)CH₃), C₁-C₆, preferably C₁-C₄, hydroxyalkoxy (e.g. —O—CH₂CH₂OH or —O—CH₂CH₂CH₂OH), C₁-C₆, preferably C₁-C₄, alkoxycarbonyl (e.g. methoxycarbonyl or ethoxycarbonyl), C₃-C₈, preferably C₅-C₆, cycloalkyl (e.g. cyclopropyl, cyclobutyl, cyclopentyl or cyclohexyl), —NR¹¹R¹², —COOR²⁹, —CONR³⁰R³¹, —SO₂NR³²R³³, —NR³⁴SO₂R³⁵, Z′R⁶⁹, (CH₂)₁₋₆NR⁷⁰R⁷¹, SO₂R⁷², NR⁷³CONR⁷⁴SO₂R⁷⁵ or M(CH₂)₁₋₆COOR⁷⁶ wherein M represents a bond, O, S, SO, SO₂, and a group >NR⁷⁷.

In an embodiment of the invention, R⁷ and R⁸ together with the nitrogen atom to which they are attached form a 5- to 6-membered saturated heterocyclic ring that optionally further comprises a ring nitrogen atom, the heterocyclic ring being optionally substituted with at least one substituent (e.g. one or two substituents independently) selected from halogen, hydroxyl, C₁-C₄ alkoxy, C₁-C₄ alkylthio, C₁-C₄ hydroxyalkyl, C₁-C₄ hydroxyalkoxy, C₁-C₄ alkoxycarbonyl, C₅-C₆ cycloalkyl, —NR¹¹R¹², —COOR²⁹, —CONR³⁰R³¹, —SO₂NR³²R³³ and —NR³⁴SO₂R³⁵.

In another embodiment, R⁷ and R⁸ together with the nitrogen atom to which they are attached form a 5- to 6-membered saturated heterocyclic ring that optionally further comprises a ring nitrogen atom, the heterocyclic ring being optionally substituted by —NR¹¹R¹².

R⁹ and R¹⁰ each independently represent hydrogen or a C₁-C₆, preferably C₁-C₄, alkylcarbonyl (e.g. methylcarbonyl or ethylcarbonyl), C₂-C₇ alkenyl (e.g. ethenyl, prop-1-enyl, prop-2-enyl, but-1-enyl, pent-1-enyl, hex-1-enyl, hept-1-enyl or 2-methyl-pent-2-enyl) or C₁-C₇, preferably C₁-C₄, alkyl (e.g. methyl, ethyl, n-propyl, isopropyl, n-butyl, isobutyl, tert-butyl, n-pentyl, n-hexyl and n-heptyl) group, each group being optionally substituted with at least one substituent (e.g. one, two, three or four substituents independently) selected from hydroxyl, —NR³⁶R³⁷, —COOR³⁸, —CONR³⁹R ⁴⁰, —SO₂NR⁴¹R⁴², —NR⁴³SO₂R⁴⁴, C₁-C₆, preferably C₁-C₄, alkoxy (e.g. methoxy, ethoxy, n-propoxy or n-butoxy), C₁-C₆, preferably C₁-C₄, alkylthio (e.g. methylthio, ethylthio, n-propylthio or n-butylthio), C₁-C₆, preferably C₁-C₄, alkoxycarbonyl (e.g. methoxycarbonyl or ethoxycarbonyl) and a saturated or unsaturated 3- to 10-membered ring system which may comprise at least one ring heteroatom (e.g. one, two, three or four ring heteroatoms independently) selected from nitrogen, oxygen and sulphur, the ring system in turn being optionally substituted with at least one substituent (e.g. one, two, three or four substituents independently) selected from halogen (e.g. chlorine, fluorine, bromine or iodine), hydroxyl, oxo, carboxyl, cyano, C₁-C₆, preferably C₁-C₄, alkyl (e.g. methyl, ethyl, n-propyl, isopropyl, n-butyl, isobutyl, tert-butyl, n-pentyl or n-hexyl) and C₁-C₆, preferably C₁-C₄, hydroxyalkyl (e.g. —CH₂OH, —CH₂CH₂OH, —CH₂CH₂CH₂OH or —CH(OH)CH₃).

Examples of saturated or unsaturated 3- to 10-membered ring systems R⁹ and R¹⁰, which may be monocyclic or polycyclic (e.g. bicyclic), include one or more (in any combination) of cyclopropyl, cyclobutyl, cyclopentyl, cyclohexyl, bicyclo[2.2.1]heptyl, cyclopentenyl, cyclohexenyl, phenyl, pyrrolidinyl, piperidinyl, piperazinyl, morpholinyl, thiomorpholinyl, diazabicyclo[2.2.1]hept-2-yl, pyrazolyl, thiazolidinyl, indanyl; thienyl, isoxazolyl, thiadiazolyl, pyrrolyl, furyl, thiazolyl, indolyl, imidazolyl, benzimidazolyl, triazolyl, tetrazolyl and pyridinyl.

Alternatively, R⁹ and R¹⁰ may together together with the nitrogen atom to which they are attached form a 4- to 7-membered saturated heterocyclic ring that optionally further comprises one or two ring heteroatoms independently selected from nitrogen, oxygen and sulphur (e.g. pyrrolidinyl, piperidinyl, piperazinyl, morpholinyl or thiomorpholinyl), the heterocyclic ring being optionally substituted with at least one substituent (e.g. one, two or three substituents independently) selected from —OR⁵⁴, —NR⁵⁵R⁵⁶, —(CH₂)ₜ—NR⁵⁷R⁵⁸ where t is 1, 2, 3, 4, 5 or 6, —COOR⁵⁹, —CONR⁶⁰R⁶¹, —SO₂NR⁶²R⁶³, —NR⁶⁴SO₂R⁶⁵, C₁-C₆, preferably C₁-C₄, alkoxy (e.g. methoxy, ethoxy, n-propoxy or n-butoxy), C₁-C₆, preferably C₁-C₄, alkylthio (e.g. methylthio, ethylthio, n-propylthio or n-butylthio), C₁-C₆, preferably C₁-C₄, alkoxycarbonyl (e.g. methoxycarbonyl or ethoxycarbonyl) and Z″R⁸⁰.

In an embodiment of the invention, R⁹ and R¹⁰ each independently represent hydrogen or C₁-C₄ alkyl optionally substituted with at least one substituent (e.g. one or two substituents independently) selected from hydroxyl, —NR³⁶R³⁷, —COOR³⁸, —CONR³⁹R⁴⁰, —SO₂NR⁴¹R⁴², —NR⁴³SO₂R⁴⁴, C₁-C₄ alkoxy, C₁-C₄ alkylthio, C₁-C₄ alkoxycarbonyl and a saturated or unsaturated 5- to 10-membered ring system which may comprise at least one ring heteroatom (e.g. one, two, three or four ring heteroatoms independently) selected from nitrogen, oxygen and sulphur, the ring system in turn being optionally substituted with at least one substituent (e.g. one or two substituents independently) selected from halogen, hydroxyl, oxo, carboxyl, cyano, C₁-C₄ alkyl and C₁-C₄ hydroxyalkyl.

In another embodiment, R⁹ and R¹⁰ each independently represent hydrogen or C₁-C₄ alkyl optionally substituted with at least one substituent (e.g. one or two substituents independently) selected from hydroxyl (e.g. methyl, ethyl, —CH₂CH₂OH or —CH₂CH₂CH₂OH).

R¹¹ and R¹² each independently represent hydrogen or a C₁-C₆, preferably C₁-C₄, alkylcarbonyl (e.g. methylcarbonyl or ethylcarbonyl), C₁-C₆, preferably C₁-C₄, alkoxycarbonyl (e.g. methoxycarbonyl or ethoxycarbonyl), C₂-C₇ alkenyl (e.g. ethenyl, prop-1-enyl, prop-2-enyl, but-1-enyl, pent-1-enyl, hex-1-enyl, hept-1-enyl or 2-methyl-pent-2-enyl) or C₁-C₇, preferably C₁-C₄, alkyl (e.g. methyl, ethyl, n-propyl, isopropyl, n-butyl, isobutyl, tert-butyl, n-pentyl, n-hexyl and n-heptyl) group, each group being optionally substituted with at least one substituent (e.g. one, two, three or four substituents independently) selected from hydroxyl, —NR⁴⁵R⁴⁶, —COOR⁴⁷, —CONR⁴⁸R⁴⁹, —SO₂NR⁵⁰R⁵¹, —NR⁵²SO₂R⁵³, —NR⁶⁶C(O)R⁶⁷, C₁-C₆, preferably C₁-C₄, alkoxy (e.g. methoxy, ethoxy, n-propoxy or n-butoxy), C₁-C₆, preferably C₁-C₄, alkylthio (e.g. methylthio, ethylthio, n-propylthio or n-butylthio) and C₁-C₆, preferably C₁-C₄, alkoxycarbonyl (e.g. methoxycarbonyl or ethoxycarbonyl).

In an embodiment of the invention, R¹¹ and R¹² each independently represent hydrogen or C₁-C₄ alkyl optionally substituted with at least one substituent (e.g. one or two substituents independently) selected from hydroxyl, —NR⁴⁵R⁴⁶, —COOR⁴⁷, —CONR⁴⁸R⁴⁹, —SO₂NR⁵⁰R⁵¹, —NR⁵²SO₂R⁵³, —NR⁶⁶C(O)R⁶⁷, C₁-C₄ alkylamino, di-C₁-C₄alkylamino, C₁-C₄alkoxy, C₁-C₄alkylthio and C₁-C₄alkoxycarbonyl.

In another embodiment, R¹¹ and R¹² each independently represent hydrogen or C₁-C₄ alkyl optionally substituted with at least one substituent (e.g. one or two substituents independently) selected from hydroxyl (e.g. methyl, ethyl, —CH₂CH₂OH or —CH₂CH₂CH₂OH).

Z, Z′ and Z″ independently represent a bond, O, S, SO, SO₂, >NR⁷⁸, C₁₋₆ alkylene, or a group —O(CH₂)₁₋₆—, —NR⁷⁹(CH₂)₁₋₆— or —S(O)ₚ(CH₂)₁₋₆— wherein p is 0, 1 or 2.

In an embodiment of the invention Z, Z′ and Z″ independently represent a bond, O, >NR⁷⁸ or a group —O(CH₂)₁₋₆—, preferably a bond.

R⁶⁸, R⁶⁹ and R⁸⁰ independently represent tetrazolyl or a 5- to 6-membered, preferably 5-membered, heterocyclic ring comprising from 1 to 4, preferably 1 to 3 and more preferably 2 to 3, heteroatoms selected from nitrogen, oxygen and sulphur, which heterocyclic ring is substituted by at least one substituent (e.g. one two or three substituents independently) selected from hydroxyl, ═O, and ═S, and which heterocyclic ring may further be optionally substituted by at least one substituent selected from halogen (e.g. chlorine, fluorine, bromine or iodine), nitro, cyano, —SO₂C₁₋₆ alkyl, C₁₋₆ alkoxycarbonyl, and a C₁₋₆, preferably C₁₋₄, alkyl group which alkyl group can be optionally substituted by at least one substituent (e.g. one, two or three substituents independently) selected from halogen (e.g. chlorine, fluorine, bromine or iodine) and hydroxyl.

R¹³, R¹⁴, R¹⁵, R¹⁶, R¹⁷, R¹⁸, R¹⁹, R²⁰ and R²¹ each independently represent hydrogen or C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one substituent (e.g. one, two or three substituents independently) selected from hydroxyl, halogen (e.g. chlorine, fluorine, bromine or iodine) and C₁-C₆, preferably C₁-C₄, alkoxy (e.g. methoxy, ethoxy, n-propoxy or n-butoxy).

R²², R²³, R²⁴, R²⁵, R²⁶, R²⁷, R²⁸, R²⁹, R³⁰, R³¹, R³², R³³, R³⁴ and R³⁵ each independently represent hydrogen or C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one substituent (e.g. one, two or three substituents independently) selected from hydroxyl, halogen (e.g. chlorine, fluorine, bromine or iodine) and C₁-C₆, preferably C₁-C₄, alkoxy (e.g. methoxy, ethoxy, n-propoxy or n-butoxy).

R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵² and R⁵³ each independently represent hydrogen or C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one substituent (e.g. one, two or three substituents independently) selected from hydroxyl, halogen (e.g. chlorine, fluorine, bromine or iodine) and C₁-C₆, preferably C₁-C₄, alkoxy (e.g. methoxy, ethoxy, n-propoxy or n-butoxy).

R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶ and R⁶⁷ each independently represent hydrogen or C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one substituent (e.g. one, two or three substituents independently) selected from hydroxyl, halogen (e.g. chlorine, fluorine, bromine or iodine) and C₁-C₆, preferably C₁-C₄, alkoxy (e.g. methoxy, ethoxy, n-propoxy or n-butoxy).

R⁷⁰, R⁷¹, R⁷², R⁷³, R⁷⁴, R⁷⁵, R⁷⁶, R⁷⁷, R⁷⁸ and R⁷⁹ each independently represent hydrogen or C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one substituent (e.g. one, two or three substituents independently) selected from hydroxyl, halogen (e.g. chlorine, fluorine, bromine or iodine) and C₁-C₆, preferably C₁-C₄, alkoxy (e.g. methoxy, ethoxy, n-propoxy or n-butoxy).

In an embodiment of the invention:

p is 0 or 1;

R¹ represents halogen;

X is C(O)NH or NHC(O);

n is 1, 2, 3, 4 or 5;

within each grouping, CR⁵R⁶, R⁵ and R⁶ each independently represent hydrogen or C₁-C₆ alkyl;

Rrepresents an unsaturated 4- to 6-membered ring system which may comprise at least one ring heteroatom selected from nitrogen, oxygen and sulphur, the ring system being optionally substituted with at least one substituent selected from halogen and C₁-C₆ alkoxy;

R³ represents hydrogen or a group —R⁷ or —NR⁷R⁸;

q is 0;

R⁷ and R⁸ each independently represent hydrogen or C₁-C₄ alkyl optionally substituted by —NR⁹R¹⁰, or

alternatively, R⁷ and R⁸ together with the nitrogen atom to which they are attached form a 5- to 6-membered saturated heterocyclic ring that optionally further comprises a ring nitrogen atom, the heterocyclic ring being optionally substituted by —NR¹¹R¹² or carboxyl;

R⁹ and R¹⁰ each independently represent hydrogen or C₁-C₄ alkyl optionally substituted with at least one substituent selected from hydroxyl; and

R¹¹ and R¹² each independently represent hydrogen or C₁-C₄ alkyl optionally substituted with at least one substituent selected from hydroxyl.

In a further embodiment of the invention:

p is 0 or 1;

R¹ represents chlorine;

X is C(O)NH or NHC(O);

n is 2;

within each grouping, CR⁵R⁶, R⁵ and R⁶ each independently represent hydrogen or methyl;

R² represents phenyl optionally substituted with one or two substituents selected from chlorine and methoxy;

R³ represents hydrogen or a group —R⁷ or —NR⁷R⁸;

q is 0;

R⁷ and R⁸ each independently represent hydrogen or C₁-C₄ alkyl optionally substituted by —NR⁹R¹⁰, or

alternatively, R⁷ and R⁸ together with the nitrogen atom to which they are attached form a 5- to 6-membered saturated heterocyclic ring that optionally further comprises a ring nitrogen atom, the heterocyclic ring being optionally substituted by —NR¹¹R¹² or carboxyl;

R⁹ and R¹⁰ each independently represent hydrogen or C₁-C₄ alkyl optionally substituted with at least one substituent selected from hydroxyl; and

R¹¹ and R¹² each independently represent hydrogen or C₁-C₄ alkyl optionally substituted with at least one substituent selected from hydroxyl.

In an embodiment of the invention the compound of formula (I) is selected from
6-Chloro-2-methyl-N-[(2R)-2-phenylpropyl]-5-quinolinecarboxamide,
6-Chloro-2-methyl-N-[(2S)-2-phenylpropyl]-5-quinolinecarboxamide,
(βR)-N-[6-Chloro-2-[methyl [3-(methylamino)propyl]amino]-5-quinolinyl]-β-methyl-benzenepropanamide,
(βR)-N-[6-Chloro-2-(1-piperazinyl)-5-quinolinyl]-β-methyl-benzenepropanamide,
6-Chloro-2-methyl-N-(2-phenylethyl)-5-quinolinecarboxamide,
(βR)-N-[6-Chloro-2-[3-(ethylamino)propyl]-5-quinolinyl]-β-methyl-benzenepropanamide,
(βR)-N-[6-Chloro-2-[3-[(3-hydroxypropyl)amino]propyl]-5-quinolinyl]-β-methyl-benzenepropanamide,
3,4-Dichloro-α-methyl-N-5-quinolinyl-benzenepropanamide,
(βR)-N-[6-Chloro-2-[[2-[(2-hydroxyethyl)amino]ethyl]amino]-5-quinolinyl]-β-methyl-benzenepropanamide,
2-Chloro-N-[6-chloro-2-(1-piperazinyl)-5-quinolinyl]-benzenepropanamide,
2,4-Dichloro-N-[6-chloro-2-(1-piperazinyl)-5-quinolinyl]-benzenepropanamide,
4-Chloro-N-[6-chloro-2-(1-piperazinyl)-5-quinolinyl]-benzenepropanamide,
(βR)-N-[2-[(3S)-3-Amino-1-pyrrolidinyl]-6-chloro-5-quinolinyl]-β-methyl-benzenepropanamide,
N-[6-Chloro-2-(1-piperazinyl)-5-quinolinyl]-2-methoxy-benzenepropanamide,
(βR)-N-[6-Chloro-2-[(3S)-3-[(3-hydroxypropyl)amino]-1-pyrrolidinyl]-5-quinolinyl]-β-methyl-benzenepropanamide,
(βR)-N-[6-Chloro-2-[(3S)-3-[(2-hydroxyethyl)amino]-1-pyrrolidinyl]-5-quinolinyl]-β-methyl-benzenepropanamide,
N-[6-Chloro-2-(1-piperazinyl)-5-quinolinyl]-benzenepropanamide,
N-[2-[(3S)-3-Amino-1-pyrrolidinyl]-6-chloro-5-quinolinyl]-2-chlorobenzenepropanamide,
2-Chloro-N-[6-chloro-2-[(3S)-3-[(2-hydroxyethyl)amino]-1-pyrrolidinyl]-5-quinolinyl]-benzenepropanamide,
1-[6-Chloro-5-[[3-(2-chlorophenyl)-1-oxopropyl]amino]-2-quinolinyl]-4-piperidinecarboxylic acid,
2-[(3S)-3-Amino-1-pyrrolidinyl]-6-chloro-N-[2-(2-chlorophenyl)ethyl]-5-quinolinecarboxamide,
6-Chloro-N-[2-(2-chlorophenyl)ethyl]-2-[(3S)-3-[(2-hydroxyethyl)amino]-1-pyrrolidinyl]-5-quinolinecarboxamide,
1-[6-Chloro-5-[[[2-(2,6-dichlorophenyl)ethyl]amino]carbonyl]-2-quinolinyl]-4-piperidinecarboxylic acid,
1-[6-Chloro-5-[[[2-(2-chlorophenyl)ethyl]amino]carbonyl]-2-quinolinyl]-4-piperidinecarboxylic acid,
1-[6-Chloro-5-[[(2,2-diphenylethyl)amino]carbonyl]-2-quinolinyl]-4-piperidinecarboxylic acid,
1-[6-Chloro-5-[[(2-phenylethyl)amino]carbonyl]-2-quinolinyl]-4-piperidinecarboxylic acid,
1-[6-Chloro-5-[[[2-(2-fluorophenyl)ethyl]amino]carbonyl]-2-quinolinyl]-4-piperidinecarboxylic acid,
1-[6-Chloro-5-[[[2-(2-methylphenyl)ethyl]amino]carbonyl]-2-quinolinyl]-4-piperidinecarboxylic acid,
1-[6-Chloro-5-[[[(2S)-2-phenylpropyl]amino]carbonyl]-2-quinolinyl]-4-piperidinecarboxylic acid,
6-Chloro-N-[2-(2-chlorophenyl)ethyl]-2-[4-(1,5-dihydro-5-oxo-4H-1,2,4-triazol-4-yl)-1-piperidinyl]-5-quinolinecarboxamide, and
1-[6-Chloro-5-[[[2-(4-chlorophenyl)ethyl]amino]carbonyl]-2-quinolinyl]-4-piperidinecarboxylic acid
   and all their pharmaceutically acceptable salts and solvates.

Suitable pharmaceutically acceptable salts of compounds of formula (I) include acid addition salts such as methanesulphonate, fumarate, hydrochloride, hydrobromide, citrate, maleate and salts formed with phosphoric and sulphuric acid. In another aspect, where the compound is sufficiently acidic, suitable salts include base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. There may be more than one cation or anion depending on the number of charged functions and the valency of the cations or anions. A preferred pharmaceutically acceptable salt is a hydrochloride salt.

Examples of compounds of formula (I) and pharmaceutically acceptable salts or solvates thereof, include:
6-Chloro-2-methyl-N-[(2R)-2-phenylpropyl]-5-quinolinecarboxamide, hydrochloride,
6-Chloro-2-methyl-N-[(2S)-2-phenylpropyl]-5-quinolinecarboxamide, hydrochloride,
(βR)-N-[6-Chloro-2-[methyl[3-(methylamino)propyl]amino]-5-quinolinyl]-β-methyl-benzenepropanamide ditrifluoroacetate,
(βR)-N-[6-Chloro-2-(1-piperazinyl)-5-quinolinyl]-β-methyl-benzenepropanamide,
6-Chloro-2-methyl-N-(2-phenylethyl)-5-quinolinecarboxamide,
(βR)-N-[6-Chloro-2-[3-(ethylamino)propyl]-5-quinolinyl]-β-methyl-benzenepropanamide dihydrochloride,
(βR)-N-[6-Chloro-2-[3-[(3-hydroxypropyl)amino]propyl]-5-quinolinyl]-β-methyl-benzenepropanamide,
3,4-Dichloro-α-methyl-N-5-quinolinyl-benzenepropanamide,
(βR)-N-[6-Chloro-2-[[2-[(2-hydroxyethyl)amino]ethyl]amino]-5-quinolinyl]-β-methyl-benzenepropanamide dihydrochloride,
2-Chloro-N-[6-chloro-2-(1-piperazinyl)-5-quinolinyl]-benzenepropanamide dihydrochloride,
2,4-Dichloro-N-[6-chloro-2-(1-piperazinyl)-5-quinolinyl]-benzenepropanamide dihydrochloride,
4-Chloro-N-[6-chloro-2-(1-piperazinyl)-5-quinolinyl]-benzenepropanamide dihydrochloride,
(βR)-N-[2-[(3S)-3-amino-1-pyrrolidinyl]-6-chloro-5-quinolinyl]-β-methyl-benzenepropanamide,
N-[6-Chloro-2-(1-piperazinyl)-5-quinolinyl]-2-methoxy-benzenepropanamide,
(βR)-N-[6-Chloro-2-[(3S)-3-[(3-hydroxypropyl)amino]-1-pyrrolidinyl]-5-quinolinyl]-β-methyl-benzenepropanamide,
(βR)-N-[6-Chloro-2-[(3S)-3-[(2-hydroxyethyl)amino]-1-pyrrolidinyl]-5-quinolinyl]-β-methyl-benzenepropanamide, dihydrochloride,
N-[6-Chloro-2-(1-piperazinyl)-5-quinolinyl]-benzenepropanamide,
N-[2-[(3S)-3-Amino-1-pyrrolidinyl]-6-chloro-5-quinolinyl]-2-chlorobenzenepropanamide,
2-Chloro-N-[6-chloro-2-[(3S)-3-[(2-hydroxyethyl)amino]-1-pyrrolidinyl]-5-quinolinyl]-benzenepropanamide,
1-[6-Chloro-5-[[3-(2-chlorophenyl)-1-oxopropyl]amino]-2-quinolinyl]-4-piperidinecarboxylic acid, potassium salt,
2-[(3S)-3-Amino-1-pyrrolidinyl]-6-chloro-N-[2-(2-chlorophenyl)ethyl]-5-quinolinecarboxamide,
6-Chloro-N-[2-(2-chlorophenyl)ethyl]-2-[(3S)-3-[(2-hydroxyethyl)amino]-1-pyrrolidinyl]-5-quinolinecarboxamide,
1-[6-Chloro-5-[[[2-(2,6-dichlorophenyl)ethyl]amino]carbonyl]-2-quinolinyl]-4-piperidinecarboxylic acid,
1-[6-Chloro-5-[[[2-(2-chlorophenyl)ethyl]amino]carbonyl]-2-quinolinyl]-4-piperidinecarboxylic acid,
1-[6-Chloro-5-[[(2,2-diphenylethyl)amino]carbonyl]-2-quinolinyl]-4-piperidinecarboxylic acid, acetate,
1-[6-Chloro-5-[[(2-phenylethyl)amino]carbonyl]-2-quinolinyl]-4-piperidinecarboxylic acid,
1-[6-Chloro-5-[[[2-(2-fluorophenyl)ethyl]amino]carbonyl]-2-quinolinyl]-4-piperidinecarboxylic acid,
1-[6-Chloro-5-[[[2-(2-methylphenyl)ethyl]amino]carbonyl]-2-quinolinyl]-4-piperidinecarboxylic acid,
1-[6-Chloro-5-[[[(2S)-2-phenylpropyl]amino]carbonyl]-2-quinolinyl]-4-piperidinecarboxylic acid,
6-Chloro-N-[2-(2-chlorophenyl)ethyl]-2-[4-(1,5-dihydro-5-oxo-4H-1,2,4-triazol-4-yl)-1-piperidinyl]-5-quinolinecarboxamide, and
1-[6-Chloro-5-[[[2-(4-chlorophenyl)ethyl]amino]carbonyl]-2-quinolinyl]-4-piperidinecarboxylic acid.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

The present invention also extends to suitable prodrugs of compounds of formula (I), i.e. compounds which are hydrolysed in vivo to form compounds of formula (I). Thus for example where compounds of formula (I) include a carboxy group, these may be in the form of pharmaceutically acceptable esters or amides. Suitable pharmaceutically acceptable esters of formula (I) for carboxy groups include C₁₋₆alkyl esters, for example methyl or ethyl; C₁₋₆alkoxymethyl esters, for example methoxymethyl; C₁₋₆alkanoyloxymethyl esters, for example pivaloyloxymethyl; phthalidyl esters; C₃₋₈cycloalkoxycarbonyloxyC₁₋₆alkyl esters, for example 1-cyclohexylcarbonyloxyethyl; 1,3-dioxolan-2-ylmethyl esters, for example 5-methyl-1,3-dioxolan-2-ylmethyl; C₁₋₆alkoxycarbonyloxyethyl esters, for example 1-methoxycarbonyloxyethyl; aminocarbonylmethyl esters and mono- or di-N-(C₁₋₆alkyl) versions thereof, for example N,N-dimethylaminocarbonylmethyl esters and N-ethylaminocarbonylmethyl esters; and may be formed at any carboxy group in the compounds of this invention. An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include C₁₋₆alkanoyl esters, for example acetyl esters; and benzoyl esters wherein the phenyl group may be substituted with aminomethyl or N-substituted mono- or di-C₁₋₆alkyl aminomethyl, for example 4-aminomethylbenzoyl esters and 4-N,N-dimethylaminomethylbenzoyl esters. Pharmaceutically acceptable amides are similarly in-vivo hydrolysable to yield the parent acid, and include C₁₋₆alkylamides such as acetamide.

The present invention further provides a process for the preparation of a compound of formula (I) as defined above, or a pharmaceutically acceptable salt or solvate thereof, which comprises

(a) reacting a compound of formula

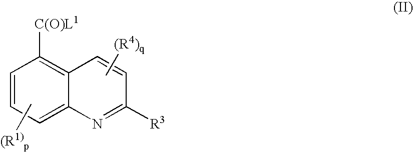

wherein L¹ represents a leaving group (e.g. hydroxyl or halogen) and p, q, R¹, R³ and R⁴ are as defined in formula (I), with a compound of formula

H₂N—(CR⁵R⁶)ₙ—R² (III)

wherein n, R², R⁵ and R⁶ are as defined in formula (I); or

(b) reacting a compound of formula

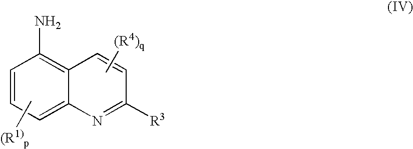

wherein p, q, R¹, R³ and R⁴ are as defined in formula (I), with a compound of formula

L²C(O)—(CR⁵R⁶)ₙ—R² (V)

wherein L² represents a leaving group (e.g. hydroxyl or halogen) and n, R², R⁵ and R⁶ are as defined in formula (I); or

(c) when R³ represents a group —NR⁷R⁸, reacting a compound of formula

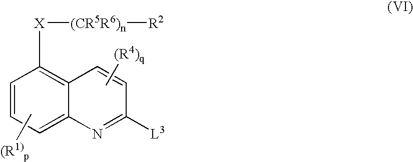

wherein L³ is a leaving group (e.g. chloride, bromide, fluoride, iodide, paratoluenesulphonate or methanesulphonate) and n, p, q, X, R¹, R², R⁴, R⁵ and R⁶ are as defined in formula (I), with a compound of formula (VII), H—NR⁷R⁸, wherein R⁷ and R⁸ are as defined in formula (I); or

(d) when R³ represents a group R⁷ where R⁷ is an optionally substituted C₃-C₁₀ alkyl group, reacting a compound of formula (VI) as defined in (c) above with a compound of formula

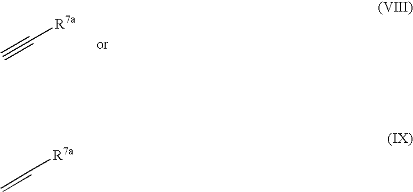

wherein R^{7a} represents a C₁-C₈ alkyl group optionally substituted as defined for R⁷ in formula (I), optionally followed by a hydrogenation reaction; or

(e) when R³ represents a group R⁷ where R⁷ is —(CH₂)₂NR⁹R¹⁰, reacting a compound of formula (VI) as defined in (c) above with a compound of formula

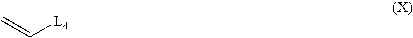

wherein L⁴ is a leaving group (eg. trialkyltin, dialkylboron or zinc), followed by reaction with a compound of formula (XI), HNR⁹R¹⁰, wherein R⁹ and R¹⁰ are as defined in formula (I); or

(f) when R³ represents a group R⁷ where R⁷ is —CH₂NR⁹R¹⁰, reacting a compound of formula (VI) as defined in (c) above with a compound of formula (X) as defined in (e) above, followed by an oxidation reaction and then by reaction with a compound of formula (XI) as defined in (e) above under reductive amination conditions; or

(g) when R³ represents a group R⁷ZR⁶⁸ or NR⁷R⁸ wherein R⁷ and/or R⁸ are substituted by a group Z′R⁶⁹ or R⁷ and R⁸ together with the nitrogen atom to which they are attached form a 4- to 7-membered heterocyclic ring substituted by a group Z′R⁶⁹, and R⁶⁸ or R⁶⁹ is tetrazolyl, reacting a group of formula (XII) or (XIII)

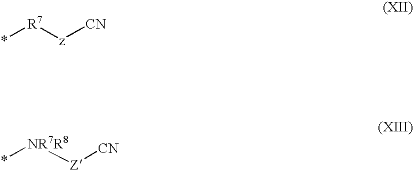

with a compound of formula GN₃, wherein G is sodium, a trialkylsilyl, an alkyltin or ammonium, to yield a group of formula (I) wherein R⁷R⁸, Z, Z′ are as defined in formula (I); or

(h) when R³ represents a group R⁷ZR⁶⁸ or NR⁷R⁸ wherein R⁷ and/or R⁸ are substituted by a group Z′R⁶⁹ or R⁷ and R⁸ together with the nitrogen atom to which they are attached form a 4- to 7-membered heterocyclic ring substituted by a group Z′R⁶⁹, and R⁶⁸ or R⁶⁹ is group of formula

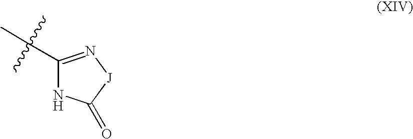

reacting a compound of formula XII or XIII wherein XII or XIII are as defined in (g) above with hydroxylamine, followed by treatment with 1,1′-thiocarbonyldiimidazole and subsequent treatment with silica gives a group of formula (XIV) wherein J is S, alternatively reacting a compound of formula XII or XIII wherein XIII or XIII are as defined in (g) above with hydroxylamine, followed by treatment with a suitable chloroformate gives a group of formula (XIV) wherein J is O; or

(i) when R³ represents a group R⁷ZR⁶⁸ or NR⁷R⁸ wherein R⁷ and/or R⁸ are substituted by a group Z′R⁶⁹ or R⁷ and R⁸ together with the nitrogen atom to which they are attached form a 4- to 7-membered heterocyclic ring substituted by a group Z′R⁶⁹, and R⁶⁸ or R⁶⁹ is

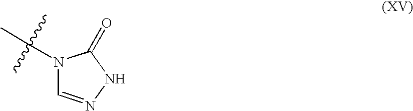

reacting a compound of formula XVI or XVII

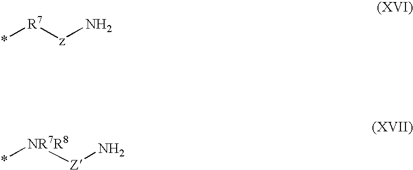

with a source of phosgene followed by treatment with formyl hydrazine and subsequent treatment with base;
and optionally after (a), (b), (c), (d), (e), (f), (g), (h) or (i) carrying out one or more of the following:
converting the compound obtained to a further compound of the invention
forming a pharmaceutically acceptable salt or solvate of the compound.

In processes (a) and (b) the coupling reaction is conveniently carried out in an organic solvent such as acetone, dichloromethane, N,N-dimethylformamide or 1-methyl-2-pyrrolidinone. If L¹ or L² represent a hydroxyl group, it may be necessary or desirable to use a coupling agent such as bromo-tris-pyrrolidino-phosphonium hexafluorophosphate (PyBroP). If L¹ or L² are chloride, such compounds may be conveniently prepared by treatment of the corresponding carboxylic acid derivative under standard conditions (such as thionyl chloride in dichloromethane with additional N,N-dimethylformamide) and used in a solvent such as acetone or dichloromethane with a suitable base such as potassium carbonate or triethylamine.

In process (c) the reaction may be performed in an organic solvent such as acetonitrile, N,N-dimethylformamide or 1-methyl-2-pyrrolidinone, and in the presence of a suitable base such as sodium hydride, triethylamine or potassium carbonate.

In process (d), if the compound of formula (VI) is reacted with a compound of formula (VIII), then the reaction is conveniently carried out in an organic solvent such as acetonitrile, e.g. at ambient temperature (20° C.), in the presence of catalytic bistriphenylphosphine dichloride palladium (0), copper (I) iodide and a base (e.g. triethylamine). The subsequent hydrogenation reaction may use hydrogen gas with a catalyst such as 5% rhodium on carbon in a solvent, for example, ethyl acetate or ethanol, and at a pressure of 3 bar.

Alternatively, if the compound of formula (VI) is reacted with a compound of formula (IX), then it is preferred if the compound of formula (IX) is pre-treated by reaction with a hydroborating reagent (e.g. 9-borabicyclo[3.3.1]nonane or catecholborane) in an organic solvent such as diethyl ether or tetrahydrofuran at a temperature in the range from, e.g. 0° C. to 80° C., in particular from 60° C. to 70° C., for about 2 to 3 hours. The pre-treated compound is then reacted with the compound of formula (VI) in the presence of a suitable base (e.g. sodium hydroxide or tri-potassium orthophosphate) and a palladium catalyst (e.g. dichloro[1,1′-bis(diphenylphosphino)ferrocene]palladium (II) dichloromethane adduct, or tetrakis(triphenylphosphine)palladium (0)), typically at a temperature in the range from 25° C. to 90° C., particularly from 60° C. to 70° C., for about 2 to 24 hours.

In process (e), the reaction with the vinyl compound of formula (X) may conveniently be carried out in a solvent such as N,N-dimethylformamide and in the presence of catalytic dichlorobis(triphenylphosphine) palladium, at elevated temperature, e.g. at about 70° C. The subsequent addition reaction with the compound of formula (XI) may be performed under acidic or basic conditions, for example, in acetic acid in a solvent such as methanol or isopropanol at elevated temperature, e.g. at about 100° C.

In process (f), the reaction of the vinyl compound of formula (X) may be performed by procedures analogous to those outlined in the previous paragraph on process (e). The subsequent oxidation reaction may be carried out under standard conditions, for example, by using ozone followed by treatment with dimethylsulfide or triphenylphosphine in a suitable solvent such as dichloromethane, or, by using osmium tetroxide and sodium periodate in a suitable solvent such as 1,4-dioxane and water. The reductive amination step may be conveniently carried out in the presence of a reducing agent such as sodium cyanoborohydride, triacetoxyborohydride or sodium borohydride, in a polar solvent such as methanol, ethanol or dichloromethane either alone or in combination with acetic acid.

In process (g), the compound of formula XII or XIII is treated with a compound of the formula GN₃ in a solvent (such as toluene, N,N-dimethylformamide or 1-methyl-2-pyrrolidinone) optionally in the presence of catalyst (such as dibutyltin oxide) at a temperature in the range from 70° C. to 120° C.

In process (h), the compound of formula XII or XIII wherein XII or XIII are defined as in (g) and J=O, is treated with hydroxylamine in a suitable solvent (such as methanol or ethanol) at a temperature in the range from 20° C. to 130° C. The resulting intermediate is treated with a suitable chloroformate (such as 2-ethylhexylchloroformate) in a suitable solvent (such as xylene) and heated at a temperature in the range from 70° C. to 150° C. to give the desired compounds of the formula (I). Alternatively, when J=S, treatment of the hydroxylamine adduct with 1,1′-thiocarbonyldiimidazole in a suitable solvent (such as tetrahydrofuran) and addition of silica yields the desired compounds of the formula (I).

In process (i), the compound of formula XVI or XVII is treated with phosgene or a phosgene equivalent (such as triphosgene) in a suitable solvent (such as dichloromethane) with a suitable base (such as triethylamine). The resulting compound is further treated with formyl hydrazine and the product subsequently treated with a base (such as potassium hydroxide) in a suitable solvent (such as methanol) at a temperature in the range from 50° C. to 130° C. to give the desired compounds of the formula (I).

Compounds of formulae (II), (III), (IV), (V), (VI), (VII), (VIII), (IX), (X), (XI), (XII) and (XIII) are either commercially available, are known in the literature or may be prepared using known techniques.

Compounds of formula (I) can be converted into further compounds of formula (I) using standard procedures. For example, compounds of formula (I) in which R¹ represents a halogen atom may be converted to a corresponding compound of formula (I) in which R¹ represents a C₁-C₆ alkyl group by reaction with an alkyl Grignard reagent (e.g. methyl magnesium bromide) in the presence of a catalyst such as [1,3-bis(diphenylphosphino)propane]dichloronickel (II) in a solvent such as tetrahydrofuran.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (I) may involve, at various stages, the addition and removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

The compounds of formula (I) above may be converted to a pharmaceutically acceptable salt or solvate thereof, preferably an acid addition salt such as a hydrochloride, hydrobromide, phosphate, acetate, fumarate, maleate, tartrate, citrate, oxalate, methanesulphonate or p-toluenesulphonate, or an alkali metal salt such as a sodium or potassium salt. Other pharmaceutically acceptable salts, as well as prodrugs such as pharmaceutically acceptable esters and pharmaceutically acceptable amides may be prepared using conventional methods.

The compounds of the present invention are advantageous in that they possess pharmacological activity. They are therefore indicated as pharmaceuticals for use in the treatment of rheumatoid arthritis, osteoarthritis, psoriasis, allergic dermatitis, asthma, chronic obstructive pulmonary disease (COPD), hyperresponsiveness of the airway, septic shock, glomerulonephritis, inflammatory bowel disease, Crohn's disease, ulcerative colitis, atherosclerosis, growth and metastases of malignant cells, myoblastic leukaemia, diabetes, Alzheimer's disease, meningitis, osteoporosis, burn injury, ischaemic heart disease, stroke, varicose veins, sarcoidosis, rhinitis, acute and chronic pain, multiple sclerosis, myeloma, bone loss associated with malignancy and inflammatory and neurodegenerative diseases of the eye such as scleritis, episcleritis, uveitis, Sjogrens syndrome-keratoconjuctivitis, sclerokeratitis, optic neuritis, diabetic retinopathy, retinitis pigmentosa, and antimalarial-induced retinopathy. They are also advantageous in the treatment of infectious diseases, e.g. anthrax, in particular inflammatory disease caused or exacerbated by bacterial toxins.

Accordingly, the present invention provides a compound of formula (I), or a pharmaceutically acceptable salt or solvate thereof, as hereinbefore defined for use in therapy.

In another aspect, the invention provides the use of a compound of formula (I), or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention further provides a method of effecting immunosuppression (e.g. in the treatment of rheumatoid arthritis, osteoarthritis, irritable bowel disease, atherosclerosis or psoriasis) which comprises administering a therapeutically effective amount of a compound of formula (I), or a pharmaceutically acceptable salt or solvate thereof, as hereinbefore defined to a patient.

The invention also provides a method of treating an obstructive airways disease (e.g. asthma or COPD) which comprises administering to a patient a therapeutically effective amount of a compound of formula (I), or a pharmaceutically acceptable salt or solvate thereof, as hereinbefore defined to a patient.

For the above-mentioned therapeutic uses the dosage administered will, of course, vary with the compound employed, the mode of administration, the treatment desired and the disorder indicated. The daily dosage of the compound of formula (I)/salt/solvate (“active ingredient”) may be in the range from 0.001 mg/kg to 30 mg/kg.

The compounds of formula (I) and pharmaceutically acceptable salts or solvates thereof may be used on their own but will generally be administered in the form of a pharmaceutical composition in which the formula (I) compound/salt/solvate (“active ingredient”) is in association with a pharmaceutically acceptable adjuvant, diluent or carrier. Depending on the mode of administration, the pharmaceutical composition will preferably comprise from 0.05 to 99% w (percent by weight), more preferably from 0.10 to 70% w, of active ingredient, and, from 1 to 99.95% w, more preferably from 30 to 99.90% w, of a pharmaceutically acceptable adjuvant, diluent or carrier, all percentages by weight being based on total composition.

Thus, the present invention also provides a pharmaceutical composition comprising a compound of formula (I), or a pharmaceutically acceptable salt or solvate thereof, as hereinbefore defined in association with a pharmaceutically acceptable adjuvant, diluent or carrier.

The pharmaceutical composition of the invention may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally.

The invention further relates to combination therapies for the treatment of any one of rheumatoid arthritis, osteoarthritis, osteoporosis, psoriasis, inflammatory bowel diseases, COPD, asthma, allergic rhinitis or cancer or the neurodegenerative diseases such as multiple sclerosis, Alzheimer's disease or stroke.

For the treatment of rheumatoid arthritis, the compounds of the invention may be combined with “biological agents” such as TNF-α inhibitors such as anti-TNF monoclonal antibodies (such as Remicade, CDP-870 and Humira) and TNF receptor immunoglobulin molecules (such as Enbrel.reg.). IL-1 receptor antagonist (such as Anakinra) and IL-1 trap, IL-18 receptor, anti-IL-6 Ab, anti-CD20 Ab, anti-IL-15 Ab and CTLA4Ig.

Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin. The COX-2 inhibitors (such as meloxicam, celecoxib, rofecoxib, valdecoxib and etoricoxib) and the cylco-oxygenase inhibiting nitric oxide donors (CINOD's) and the “disease modifying agents” (DMARDs) such as methotrexate, sulphasalazine, cyclosporine A, lefunomide; ciclesonide; hydroxychloroquine, d-penicillamine, auranofin or parenteral or oral gold.

The present invention still further relates to the combination of a compound of the invention together with a leukotriene biosynthesis inhibitor, 5-lipoxygenase (5-LO) inhibitor or 5-lipoxygenase activating protein (FLAP) antagonist selected from the group consisting of zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2n cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of the invention together with a receptor antagonists for leukotrienes LTB₄, LTC₄, LTD₄, and LTE₄ selected from the group consisting of the phenothiazin-3-ones such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a PDE4 inhibitor including inhibitors of the isoform PDE4D.

The present invention still further relates to the combination of a compound of the invention together with a antihistaminic H₁ receptor antagonists including cetirizine, loratadine, desloratadine, fexofenadine, astemizole, azelastine, and chlorpheniramine.

The present invention still further relates to the combination of a compound of the invention together with a gastroprotective H₂ receptor antagonist or the proton pump inhibitors (such as omeprazole)

The present invention still further relates to the combination of a compound of the invention together with an α₁- and α₂-adrenoceptor agonist vasoconstrictor sympathomimetic agent, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents including ipratropium bromide; tiotropium bromide; oxitropium bromide; pirenzepine; and telenzepine.

The present invention still further relates to the combination of a compound of the invention together with a β₁- to β₄-adrenoceptor agonists including metaproterenol isoproterenol, isoprenaline, albuterol, salbutamol, formoterol, salmeterol, terbutaline, orciprenaline, bitolterol mesylate, and pirbuterol; or methylxanthanines including theophylline and aminophylline; sodium cromoglycate; or muscarinic receptor (M1, M2, and M3) antagonist.

The present invention still further relates to the combination of a compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR3, CXCR4 and CXCR5 (for the C—X—C family) and CX₃CR1 for the C—X₃—C family.

The present invention still further relates to the combination of a compound of the invention together with an insulin-like growth factor type I (IGF-1) mimetic.

The present invention still further relates to the combination of compound of the invention together with an inhaled glucocorticoid with reduced systemic side effects, including prednisone, prednisolone, flunisolide, triamcinolone acetonide, beclomethasone dipropionate, budesonide, fluticasone propionate, and mometasone furoate.

The present invention still further relates to the combination of a compound of the invention together with (a) tryptase inhibitors; (b) platelet activating factor (PAF) antagonists; (c) interleukin converting enzyme (ICE) inhibitors; (d) IMPDH inhibitors; (e) adhesion molecule inhibitors including VLA-4 antagonists; (f) cathepsins; (g) MAP kinase inhibitors; (h) glucose-6 phosphate dehydrogenase inhibitors; (i) kinin-B₁- and B₂-receptor antagonists; (j) anti-gout agents, e.g., colchicine; (k) xanthine oxidase inhibitors, e.g., allopurinol; (l) uricosuric agents, e.g., probenecid, sulfinpyrazone, and benzbromarone; (m) growth hormone secretagogues; (n) transforming growth factor (TGFβ); (o) platelet-derived growth factor (PDGF); (p) fibroblast growth factor, e.g., basic fibroblast growth factor (bFGF); (q) granulocyte macrophage colony stimulating factor (GM-CSF); (r) capsaicin cream; (s) Tachykinin NK₁ and NK₃ receptor antagonists selected from the group consisting of NKP-608C; SB-233412 (talnetant); and D-4418; and (t) elastase inhibitors selected from the group consisting of UT-77 and ZD-0892 (u) induced nitric oxide synthase inhibitors (iNOS) or (v) chemoattractant receptor-homologous molecule expressed on TH2 cells, (CRTH2 antagonists).

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11).

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, induced nitric oxide synthase inhibitors (iNOS inhibitors), COX-2 inhibitors such as celecoxib, valdecoxib, rofecoxib and etoricoxib, and the cylco-oxygenase inhibiting nitric oxide donors (CINOD's) analgesics (such as paracetamol and tramadol), cartilage sparing agents such as diacerein, doxycyline and glucosamine, and intra-articular therapies such as corticosteroids and hyaluronic acids such as hyalgan and synvisc.

The compounds of the invention can also be used in combination with existing therapeutic agents for the treatment of inflammatory bowel diseases (Ulcerative colitis and Crohn's disease). Suitable agents to be used include sulphasalazine, 5-amino-salicylates, the thiopurines, azathioprine and 6-mecaptorurine and corticosteroids such as budesonide.

The compounds of the present invention may also be used in combination with anticancer agents such as endostatin and angiostatin or cytotoxic drugs such as adriamycin, daunomycin, cis-platinum, etoposide, taxol, taxotere and farnesyl transferase inhibitors, VegF inhibitors, COX-2 inhibitors and antimetabolites such as methotrexate, antineoplastic agents, especially antimitotic drugs including the vinca alkaloids such as vinblastine and vincristine.

The compounds of the invention may also be used in combination with antiviral agents such as Viracept, AZT, aciclovir and famciclovir, and antisepsis compounds such as Valant.

The compounds of the present invention may also be used in combination with cardiovascular agents such as calcium channel blockers, lipid lowering agents such as statins, fibrates, beta-blockers, Ace inhibitors, Angiotensin-2 receptor antagonists and platelet aggregation inhibitors.

The compounds of the present invention may also be used in combination with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti Alzheimer's drugs such as donepezil, tacrine, COX-2 inhibitors, propentofylline or metryfonate.

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate.

The present invention will now be further explained by reference to the following illustrative examples. In the examples the NMR spectra were measured on a Varian Unity spectrometer at a proton frequency of either 300 or 400 MHz. The MS spectra were measured on either an Agilent 1100 MSD G1946D spectrometer or a Hewlett Packard HP1100 MSD G1946A spectrometer. Preparative HPLC separations were performed using a Waters Symmetry® or Xterra® column using 0.1% aqueous trifluoroacetic acid: acetonitrile, 0.1% aqueous ammonia: acetonitrile or 0.1% ammonium acetate: acetonitrile as the eluant. Microwave reactions were performed in a CEM Discover single mode microwave.

### EXAMPLE 1

### 6-Chloro-2-methyl-N-[(2R)-2-phenylpropyl]-5-quinolinecarboxamide, hydrochloride

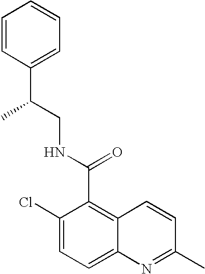

### (a) 6-Chloro-²-methyl-5-quinolinecarboxylic acid

Crotonaldehyde (1.50 mL) was added dropwise over a period of 1 hour to a mixture of 5-amino-2-chlorobenzoic acid (1.72 g), ferrous sulphate heptahydrate (0.77 g), sodium nitrobenzenesulphonate (1.23 g) and concentrated hydrochloric acid (11 mL) at 95° C. The reaction mixture was heated for a further 15 minutes then filtered whilst still hot. The resulting solid was extracted with boiling 2M aqueous hydrochloric acid solution (20 mL) and the extract combined with the filtrate. Ammonium acetate was then added to give a solution of pH 4, which was cooled in ice and the resultant precipitate collected by filtration and washed with water. The solid was dried in vacuo to give the sub-title compound (0.5 g) as a solid.

MS: APCI(+ve) 222/224 (M+1)

### (b) 6-Chloro-2-methyl-N-[(2R)-2-phenylpropyl]-5-quinolinecarboxamide, hydrochloride

To a stirred solution of 6-chloro-2-methyl-5-quinolinecarboxylic acid (Example 1(a) ) (250 mg) in dichloromethane (5 mL) at 0° C. under nitrogen, was added N,N-dimethylformamide (1 drop) and oxalyl chloride (0.4 mL). The reaction mixture was stirred at room temperature for 1 hour, then evaporated to dryness and redissolved in dichloromethane (3 mL). This solution was cooled to 0° C. and a mixture of (R)-2-phenyl-1-propylamine (152 mg) and triethylamine (1 mL) in dichloromethane (2 mL) was added dropwise. The reaction mixture was stirred at room temperature for 10 minutes then poured into saturated NaHCO₃ aq. (20 mL). The mixture was extracted with dichloromethane (3×20 mL) and the combined extracts were dried, filtered and evaporated. Purification (SiO₂, ethyl acetate:isohexane 1:1 as eluant) afforded the product which was converted to its hydrochloride salt by treatment with hydrochloric acid (4M in 1,4-dioxane) and recrystallised (ethanol/ethyl acetate) to give the title product (40 mg).

¹H NMR (400 MHz, d₆-DMSO) δ 8.87 (1H, s), 8.15 (1H, d), 7.92 (1H, d), 7.75-7.66 (1H, m), 7.58 (1H, d), 7.40-7.24 (5H, m), 3.81-3.66 (1H, m), 3.52-3.39 (1H, m), 3.13-3.02 (1H, m), 2.80 (3H, s), 1.29 (3H, d). MS: APCI(+ve) 339/341 (M+H⁺). m.p. 190-192° C.

### EXAMPLE 2

### 6-Chloro-2-methyl-N-[(2S)-2-phenylpropyl]-5-quinolinecarboxamide, hydrochloride

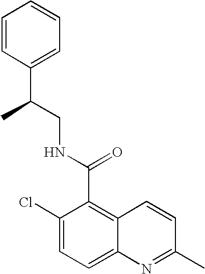

Prepared according to the method of Example 1(b), using 6-chloro-2-methyl-5-quinolinecarboxylic acid (Example 1(a)) (250 mg) and (S)-2-phenyl-1-propylamine (152 mg). Purification (SiO₂, ethyl acetate:isohexane 1:1 as eluant) afforded the product which was converted to its hydrochloride salt by treatment with hydrochloric acid (4M in 1,4-dioxane) and recrystallised (ethanol/ethyl acetate) to give the title product (38 mg).

¹H NMR (400 MHz, d₆-DMSO) δ 8.89 (1H, t), 8.18 (1H, d), 7.94 (1H, d), 7.73 (1H, d), 7.60 (1H, d), 7.38-7.25 (5H, m), 3.80-3.68 (1H, m), 3.48-3.40 (1H, m), 3.14-3.04 (1H, m), 2.81 (3H, s), 1.29 (3H, d). MS: APCI(+ve) 339/341 (M+H⁺). m.p. 182-185° C.

### EXAMPLE 3

### (βR)-N-[6-Chloro-2-[methyl[3-(methylamino)propyl]amino]-5-quinolinyl]-β-methyl-benzenepropanamide, ditrifluoroacetate

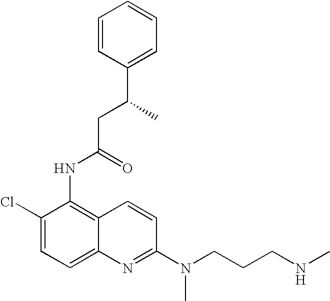

### (a) 2,6-Dichloroquinolin-5-amine

6-Chloro-5-nitroquinoline 1-oxide (4 g) was added to phosphorus oxychloride (15 mL) at 0° C. The solution was allowed to warm to room temperature and stirred for 12 hours. The excess phosphorus oxychloride was evaporated in vacuo and the residue dissolved in water (100 mL)/dichloromethane (100 mL). The layers were separated and the aqueous layer extracted with dichloromethane (2×50 mL). The combined extracts were dried over anhydrous magnesium sulfate, filtered and concentrated to give an oil. The residue was dissolved in ethanol/water (1:1, 80 mL), ammonium chloride (2.8 g) and iron (2.8 g) added. The mixture was stirred at 65° C. for 4 hours, cooled to room temperature and filtered. The resulting solid was suspended in dimethylsulphoxide (50 mL), methanol (50 mL) and aqueous hydrochloric acid added (2M, 100 mL). The resulting solid was removed by filtration and then treated with ether (50 mL) and isohexane (50 mL). Evaporation of the mixture afforded the sub-title compound as a solid (1 g).

¹H NMR (400 MHz, d₆-DMSO) δ 8.73 (1H, dd,); 7.62 (1H, d); 7.51 (1H, d); 7.13 (1H, dd); 6.36 (2H, s). MS: APCI(+ve) 213.1/214.9 (M+1)

### (b) (βR)-N-(2,6-Dichloro-5-quinolinyl)-β-methyl-benzenepropanamide

To a stirred solution of 2,6-dichloroquinolin-5-amine (prepared as described in 3(a) above) (450 mg) in N-methyl pyrrolidinone (6 mL) was added 4-N,N-dimethylaminopyridine (512 mg), (R)-3-phenylbutyric acid (515 mg) and PyBroP (2 g). The reaction mixture was heated to 50° C. for 5 hours. The mixture was cooled to room temperature and poured into water (10 mL) which was subsequently acidified to pH1 with aqueous 2M hydrochloric acid. The resulting solution was extracted with dichloromethane (3×20 mL). The combined organic extracts were dried, filtered and evaporated. Purification (SiO₂, methanol:dichloromethane 1:10 as eluant) and recrystallisation (ethyl acetate) afforded the sub-title compound as a solid (400 mg).

¹H NMR (400 MHz, d₆-DMSO) δ 10.07 (1H, s), 7.90 (2H, s), 7.63-7.55 (1H, m), 7.47 (1H, d), 7.42-7.25 (5H, m), 3.36-3.27 (1H, m), 2.83 (1H, dd), 2.73 (1H, dd), 1.34 (3H, d).

### (c) (βR)-N-[6-Chloro-2-[methyl[3-(methylamino)propyl]amino]-5-quinolinyl]-β-methyl-benzenepropanamide, ditrifluoroacetate

To a stirred solution of (βR)-N-(2,6-dichloro-5-quinolinyl)-β-methyl-benzenepropanamide (Example 3(b)) (200 mg) and potassium carbonate (385 mg) in N-methyl pyrrolidinone (2 mL) was added N,N′-dimethyl-1,3-propanediamine (570 mg). The mixture was heated at 120° C. for 1 hour after which it was cooled and poured into water. The mixture was extracted with dichloromethane and the combined extracts were dried, filtered and evaporated. Purification by HPLC (Waters Symmetry column using 25% to 95% acetonitrile in 0.1% aqueous trifluoroacetic acid) afforded the title product (250 mg).

¹H NMR (400 MHz, d₆-DMSO) δ 9.91 (1H, s), 8.50 (1H, s), 7.73-7.55 (1H, m), 7.53-7.42 (1H, m), 7.40-7.31 (3H, m), 7.30-7.23 (2H, m), 7.13-7.02 (1H, m), 3.76 (2H, t), 3.31 (1H, q), 3.18 (3H, s), 2.99-2.87 (2H, m), 2.79 (1H, dd), 2.70 (1H, dd), 2.60-2.54 (3H, m), 1.93 (2H, quint.), 1.33 (3H, d). MS: APCI(+ve) 425.2/427.2 (M+H⁺). m.p. 159-162° C.

### EXAMPLE 4

### (βR)-N-[6-Chloro-2-(1-piperazinyl)-5-quinolinyl]-β-methyl-benzenepropanamide

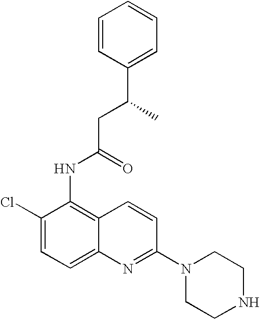

Prepared according to the method of Example 3(c), using (βR)-N-(2,6-dichloro-5-quinolinyl)-β-methyl-benzenepropanamide (Example 3(b)) (200 mg) and piperazine (580 mg). Purification (SiO₂, methanol:dichloromethane:ammonium hydroxide solution 15:85:1 as eluant) afforded the title compound as a solid (25 mg).

¹H NMR (400 MHz, d₆-DMSO) δ 9.79 (1H, s), 7.54 (1H, d), 7.44 (1H, d), 7.40-7.22 (6H, m), 7.07 (1H, d), 3.59 (4H, t), 3.38-3.25 (1H, m), 2.82-2.73 (5H, m), 2.68 (1H, dd), 1.33 (3H, d). MS: APCI(+ve) 409.2/411.2 (M+H⁺). m.p. 194-196° C.

### EXAMPLE 5

### 6-Chloro-2-methyl-N-(2-phenylethyl)-5-quinolinecarboxamide

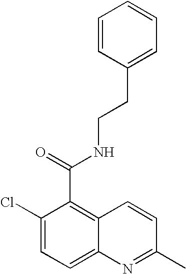

Prepared according to the method of Example 1, using 6-chloro-2-methyl-5-quinolinecarboxylic acid (Example 1(a)) (60 mg) and benzeneethanamine (33 mg). Purification (SiO₂, ethyl acetate:isohexane 3:7 as eluant) afforded the title compound as a solid (15 mg).

¹H NMR (400 MHz, d₆-DMSO) δ 8.81 (1H, t), 7.93 (1H, d), 7.73 (1H, d), 7.63 (1H, d), 7.40 (1H, d), 7.37-7.23 (5H, m), 3.65 (2H, q), 2.90 (2H, t), 2.65 (3H, s). MS: APCI(+ve) 325/327 (M+H⁺). m.p. 170-172° C.

### EXAMPLE 6

### (βR)-N-[6-Chloro-2-[3-(ethylamino)propyl]-5-quinolinyl]-β-methyl-benzenepropanamide, dihydrochloride

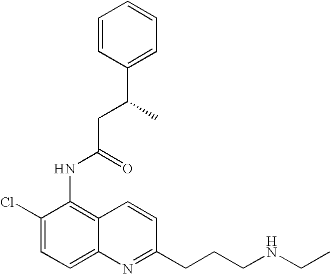

### (a) [3-[6-Chloro-5-[[(3R)-1-oxo-3-phenylbutyl]amino]-2-quinolinyl]propyl]ethylcarbamic acid, 1,1-dimethylethyl ester

9-Borabicyclo[3.3.1]nonane dimer solution (2.7 mL, 0.5 M in tetrahydrofuran) was added to ethyl(2-propenyl)-carbamic acid, 1,1-dimethylethyl ester (prepared as described in Example 7(iv) of WO 03/041707) (124 mg) at room temperature under nitrogen. The mixture was refluxed for 2 hours after which it was cooled to room temperature. Potassium phosphate (356 mg) in water (1 mL) was added and the mixture stirred for 15 minutes. (βR)-N-(2,6-Dichloro-5-quinolinyl)-β-methyl-benzenepropanamide (Example 3(b)) (200 mg) in N,N-dimethylformamide (2 mL) was added followed by tetrakis(triphenylphosphine)palladium(0) (7 mg). The reaction mixture was heated to 70° C. for 2 hours under nitrogen. On cooling to room temperature the reaction mixture was filtered through diatomaceous earth and the tetrahydrofuran removed under vacuum. The resulting mixture was poured into water and extracted with ethyl acetate. The combined organic extracts were dried, filtered and evaporated. Purification (SiO₂, ethyl acetate:isohexane 30:70 as eluant) gave the sub-title compound (250 mg).

¹H NMR (400 MHz, d₆-DMSO) δ 9.94 (1H, s), 7.86 (1H, d), 7.77 (1H, d), 7.55-7.45 (1H, m), 7.45-7.21 (6H, m), 3.40-3.26 (1H, m), 3.25-3.09 (4H, m), 2.91-2.78 (3H, m), 2.76-2.65 (1H, m), 1.98-1.90 (2H, m), 1.44-1.27 (12H, m), 1.03 (3H, t).

### (b) (βR)-N-[6-Chloro-2-[3-(ethylamino)propyl]-5-quinolinyl]-β-methyl-benzenepropanamide, dihydrochloride

[3-[6-Chloro-5-[[(3R)-1-oxo-3-phenylbutyl]amino]-2-quinolinyl]propyl]ethyl-carbamic acid, 1,1-dimethylethyl ester (Example 6(a)) was dissolved in dichloromethane (3 mL). Hydrochloric acid (HCl) in 1,4-dioxane (4M, 0.8 mL) was added and the mixture stirred for 2 hours. The resultant suspension was evaporated to dryness and recrystallised from methanol/ethyl acetate to give the title compound as a solid (170 mg).

¹H NMR (400 MHz, d₆-DMSO) δ 10.18 (1H, s), 8.90 (2H, s), 8.04 (1H, d), 7.92 (1H, d), 7.77-7.67 (1H, m), 7.52 (1H, d), 7.41-7.23 (5H, m), 3.39-3.27 (1H, m), 3.12 (2H, t), 3.02-2.81 (5H, m), 2.75 (1H, dd), 2.15 (2H, quint.), 1.34 (3H, d), 1.20 (3H, t). MS: APCI(+ve) 410/412 (M+H⁺).

### EXAMPLE 7

### (βR)-N-[6-Chloro-2-[3-[(3-hydroxypropyl)amino]propyl]-5-quinolinyl]-β-methyl-benzenepropanamide

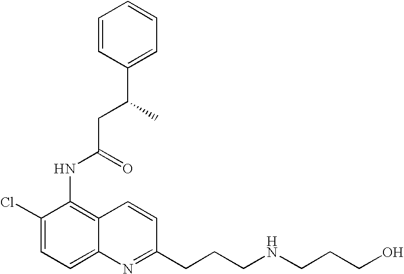

### (a) [3-[6-Chloro-5-[[(3R)-1-oxo-3-phenylbutyl]amino]-2-quinolinyl]propyl][3-[[(1,1-dimethylethyl)dimethylsilyl]oxy]propyl]-carbamic acid, 1,1-dimethylethyl ester

Prepared according to the method of example 6(a), using (βR)-N-(2,6-dichloro-5-quinolinyl)-β-methyl-benzenepropanamide (Example 3(b)) (200 mg) and [3-[[(1,1-dimethylethyl)dimethylsilyl]oxy]propyl]-2-propenyl-carbamic acid, 1,1-dimethylethyl ester (prepared as described by I. Kadota, S. Saya, Y. Yamamoto, Heterocycles, (1997), Vol. 46, pages 335-348) (221 mg). Purification (SiO₂, ethyl acetate:isohexane 1:4 as eluant) afforded the sub-title compound as a solid (300 mg).

¹H NMR (400 MHz, CDCl₃) δ 7.87 (1H, d), 7.62 (1H, d), 7.44-7.08 (5H, m), 7.15 (1H, s), 7.02 (1H, s), 3.62 (2H, t), 3.48 (1H, q), 3.28 (4H, s), 2.94-2.80 (4H, m 2.08-1.96 (2H, m), 1.74 (2H, s), 1.58 (3H, s), 1.45 (9H, s), 0.88 (9H, s), 0.04 (6H, s).

### (b) (βR)-N-[6-Chloro-2-[3-[(3-hydroxypropyl)amino]propyl]-5-quinolinyl]-β-methyl-benzenepropanamide

[3-[6-Chloro-5-[[(3R)-1-oxo-3-phenylbutyl]amino]-2-quinolinyl]propyl][3-[[(1,1-dimethylethyl)dimethylsilyl]oxy]propyl]-carbamic acid, 1,1-dimethylethyl ester (Example 7(a)) was dissolved in dichloromethane (3 mL). HCl in 1,4-dioxane (4M, 1 mL) was added and the mixture stirred for 2 hours. The resultant suspension was evaporated to dryness and the residue was dissolved in dichloromethane (10 mL) and methanol (0.5 mL) and washed with aqueous sodium hydroxide (2M, 3×5 mL). The organics were dried, filtered and evaporated. Purification (SiO₂, methanol:dichloromethane:ammonium hydroxide solution 20:80:2 as eluant) afforded the title compound as a solid (85 mg).

¹H NMR (400 MHz, d₆-DMSO) δ 9.94 (1H, s), 7.86 (1H, d), 7.77 (1H, d), 7.55-7.43 (1H, m), 7.42-7.23 (6H, m), 3.46 (2H, t), 3.40-3.21 (3H, m), 2.92 (2H, t), 2.82 (1H, dd), 2.72 (1H, dd), 2.58-2.47 (2H, m), 1.86 (2H, quint.), 1.55 (2H, quint.), 1.34 (3H, d). MS: APCI(+ve) 440/442 (M+H⁺). m.p. 118-120° C.

### EXAMPLE 8

### 3,4-Dichloro-α-methyl-N-5-quinolinyl-benzenepropanamide

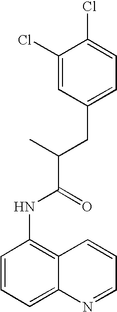

Prepared according to the method of Example 1, using 5-aminoquinoline (200 mg) and 3,4-dichloro-α-methyl-benzenepropanoic acid (652 mg). Purification by HPLC (Symmetry—0.1% aqueous ammonium acetate/acetonitrile) afforded the title compound as a solid (120 mg).

¹H NMR (400 MHz, d₆-DMSO) δ 9.94 (1H, s), 8.89 (1H, dd), 7.94 (1H, d), 7.85 (1H, d), 7.72 (1H, t), 7.63-7.54 (3H, m), 7.45 (1H, dd), 7.26 (1H, dd), 3.09-2.99 (1H, m), 2.96-2.88 (1H, m), 2.78 (1H, dd), 1.23 (3H, d). MS: APCI(+ve) 359.1/361.1 (M+H⁺). m.p. 168-170° C.

### EXAMPLE 9

### (βR)-N-[6-Chloro-2-[[2-[(2-hydroxyethyl)amino]ethyl]amino]-5-quinolinyl]-β-methyl-benzenepropanamide, dihydrochloride

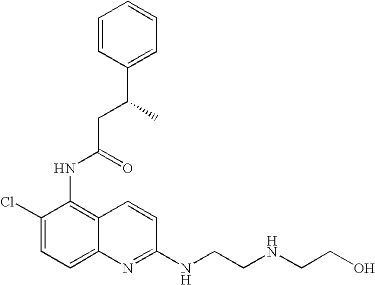

To a stirred solution of (βR)-N-(2,6-dichloro-5-quinolinyl)-β-methyl-benzenepropanamide (Example 3(b)) (200 mg) and potassium carbonate (380 mg) in N-methyl pyrrolidinone (2 mL) was added 2-[(2-aminoethyl)amino]-ethanol (580 mg). The mixture was heated at 120° C. for 3 hours after which it was cooled and poured into water. The resulting solid was isolated by filtration, dried and suspended in dichloromethane (5 mL). The suspension was then treated with di-tert-butyl dicarbonate (1.6 g) and stirred for 2 hours. The mixture was poured into water and extracted with dichloromethane (3×20 mL). The combined organic layers were dried and concentrated. Purification (SiO₂, methanol:dichloromethane:ammonium hydroxide solution 2:98:1 as eluant) yielded the desired major isomer which was then dissolved in dichloromethane (5 mL) and treated with HCl in 1,4-dioxane (4M, 1 mL) for 1 hour. The resultant suspension was evaporated to dryness and recrystallised from methanol/ethyl acetate to give the title compound as a colourless solid (50 mg).

¹H NMR (400 MHz, d₆-DMSO) δ 9.69 (1H, s), 7.87 (1H, s), 7.67 (1H, d), 7.47 (1H, d), 7.36-7.28 (4H, m), 7.26-7.19 (1H, m), 6.96-6.89 (1H, m), 3.95-3.86 (2H, m), 3.72 (2H, t), 3.34 (1H, q), 3.28 (2H, t), 3.10 (2H, t), 2.86-2.75 (1H, m), 2.75-2.64 (1H, m), 1.34 (3H, d). MS: APCI(+ve) 427/429 (M+H⁺). m.p. 178-182° C.

### EXAMPLE 10

### 2-Chloro-N-[6-chloro-2-(1-piperazinyl)-5-quinolinyl]-benzenepropanamide, dihydrochloride

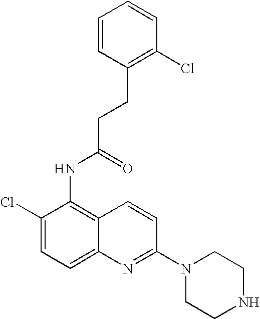

### (a) 4-(5-Amino-6-chloro-2-quinolinyl)-1-piperazinecarboxylic acid, 1,1-dimethylethyl ester

To a stirred solution of of 2,6-dichloroquinolin-5-amine (Example 3(a)) (800 mg) and potassium carbonate (2 g) in N-methyl pyrrolidinone (4 mL) was added 1-piperazinecarboxylic acid, 1,1-dimethylethyl ester (2 g). The mixture was heated at 130° C. for 4 hours after which it was cooled and poured into water. The product was collected by filtration and washed with water to give the sub-title compound as a solid (1.2 g).

¹H NMR (400 MHz, d₆-DMSO) δ 8.36 (1H, d), 7.30 (1H, d), 7.11 (1H, d), 6.82 (1H, d), 5.76 (2H, s), 3.69-3.61 (4H, m), 3.49-3.40 (4H, m), 1.48-1.34 (9H, m).

### (b) 2-Chloro-N-[6-chloro-2-(1-piperazinyl)-5-quinolinyl]-benzenepropanamide, dihydrochloride

To a stirred solution of 2-chloro-benzenepropanoic acid (204 mg) in dichloromethane (2 mL) at 0° C. under nitrogen, was added N,N-dimethylformamide (1 drop) and oxalyl chloride (0.3 mL). The reaction mixture was heated to reflux for 2 hours, then cooled, evaporated to dryness and redissolved in dichloromethane (1 mL). This solution was added to a stirred solution of 4-(5-amino-6-chloro-2-quinolinyl)-piperazinecarboxylic acid, 1,1-dimethylethyl ester (Example 10(a)) (200 mg) and potassium carbonate (380 mg) in acetone (2 mL). The reaction mixture was stirred at room temperature for 16 hours then the acetone was evaporated. The residue was redissolved in dichloromethane then poured into water and extracted with dichloromethane (3×20 mL). The combined organic extracts were dried, filtered and evaporated. The resulting solid was purified (SiO₂, methanol:dichloromethane:ammonium hydroxide solution 10:90:1 as eluant) then redissolved in methanol and treated with HCl in 1,4-dioxane (4M, 1 mL) for 1 hour. The resultant suspension was evaporated to dryness and recrystallised from methanol/ethyl acetate to give the title compound as a solid (90 mg).

¹H NMR (400 MHz, d₆-DMSO) δ 10.09 (1H, s), 9.40 (2H, s), 7.89 (1H, d), 7.83-7.69 (2H, m), 7.50-7.26 (5H, m), 4.04 (4H, s), 3.25 (4H, s), 3.08 (2H, t), 2.83 (2H, t). MS: APCI(+ve) 429 (M+H⁺). m.p. 265-270° C.

### EXAMPLE 11

### 2,4-Dichloro-N-[6-chloro-2-(1-piperazinyl)-5-quinolinyl]-benzenepropanamide, dihydrochloride

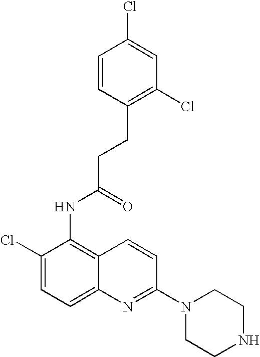

Prepared according to method of Example 10(b) using 4-(5-amino-6-chloro-2-quinolinyl)-1-piperazinecarboxylic acid, 1,1-dimethylethyl ester (Example 10(a)) (200 mg) and 2,4-dichloro-benzenepropanoic acid (242 mg). Purification by HPLC (Symmetry—0.1% aqueous ammonium acetate/acetonitrile), treatment with HCl in 1,4-dioxane (4M, 1 mL) and recrystallisation (methanol/ethyl acetate) afforded the title compound as a solid (29 mg).

¹H NMR (400 MHz, d₆-DMSO) δ 10.10 (1H, s), 9.39 (2H, s), 7.90 (1H, d), 7.83-7.67 (2H, m), 7.63 (1H, s), 7.50-7.33 (3H, m), 4.03 (4H, s), 3.25 (4H, s), 3.06 (2H, t), 2.82 (2H, t). MS: APCI(+ve) 463(M+H⁺). m.p. 200° C. (dec)

### EXAMPLE 12

### 4-Chloro-N-[6-chloro-2-(1-piperazinyl)-5-quinolinyl]-benzenepropanamide, dihydrochloride

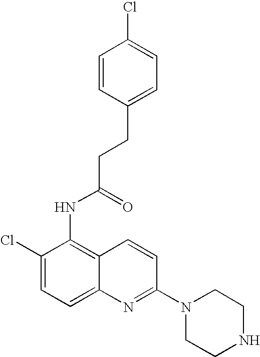

Prepared according to method of Example 10(b) using 4-(5-amino-6-chloro-2-quinolinyl)-1-piperazinecarboxylic acid, 1,1-dimethylethyl ester (Example 10(a)) (200 mg) and 4-chloro-benzenepropanoic acid (204 mg). Purification (SiO₂, methanol:dichloromethane:ammonium hydroxide solution 10:90:1 as eluant), treatment with HCl in 1,4-dioxane (4M, 1 mL) and recrystallisation (ethyl acetate/iso-hexane) afforded the title compound as a solid (17 mg).

¹H NMR (400 MHz, d₆-DMSO) δ 9.68 (1H, s), 9.30 (1H, s), 7.79 (1H, d), 7.64-7.58 (2H, m), 7.37-7.28 (4H, m), 7.23 (1H, d), 3.98 (4H, t), 3.23 (4H, s), 2.99 (2H, t), 2.78 (2H, m). MS: APCI(+ve) 429/431 (M+H⁺). m.p. 183-188° C.

### EXAMPLE 13

### (βR)-N-[2-[(3S)-3-Amino-1-pyrrolidinyl]-6-chloro-5-quinolinyl]-β-methyl-benzenepropanamide

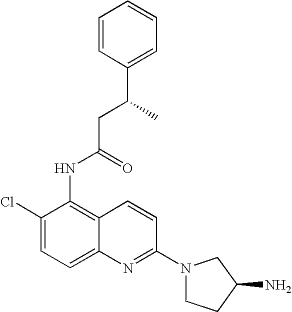

To a 10 mL microwave vial was added (βR)-N-(2,6-dichloro-5-quinolinyl)-β-methyl-benzenepropanamide (Example 3(b)) (200 mg), (3S)-3-pyrrolidinamine (145 mg), triethylamine (0.085 mL) and acetonitrile (5 mL). The vial was sealed and heated at 100° C. for 1 hour within a microwave. The reaction was cooled to room temperature and evaporated. Purification (SiO₂, methanol:dichloromethane:ammonium hydroxide solution 10:90:1 as eluant) afforded the title compound as a solid (80 mg).

¹H NMR (400 MHz, d₆-DMSO) δ 9.77 (1H, s), 7.51 (1H, d), 7.43 (1H, d), 7.39-7.30 (5H, m), 7.29-7.23 (1H, m), 6.71 (1H, d), 3.69-3.46 (4H, m), 3.38-3.26 (1H, m), 3.24-3.14 (1H, m), 2.77 (1H, dd), 2.67 (1H, dd), 2.12-2.01 (1H, m), 1.78-1.68 (1H, m), 1.33 (3H, d). MS: APCI(+ve) 409/411 (M+H⁺). m.p. 204-207° C.

### EXAMPLE 14

### N-[6-Chloro-2-(1-piperazinyl)-5-quinolinyl]-2-methoxy-benzenepropanamide

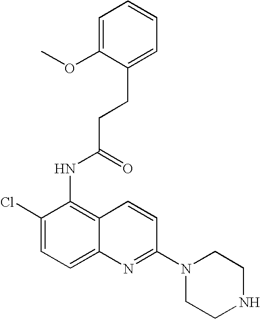

Prepared according to method of Example 10(b) using 4-(5-amino-6-chloro-2-quinolinyl)-1-piperazinecarboxylic acid, 1,1-dimethylethyl ester (Example 10(a)) (200 mg) and 2-methoxy-benzenepropanoic acid (200 mg). Purification by HPLC (Waters Symmetry column using 5% to 50% acetonitrile in 0.1% aqueous trifluoroacetic acid) and recrystallisation (methanol/ethyl acetate) afforded the title compound as a solid (25 mg).

¹H NMR (400 MHz, d₆-DMSO) δ 9.90 (1H, s), 9.10 (2H, s), 7.78 (1H, d), 7.66 (1H, d), 7.58 (1H, d), 7.34-7.19 (3H, m), 7.00 (1H, d), 6.92 (1H, t), 3.95 (4H, s), 3.83 (3H, s), 3.23 (4H, s), 2.94 (2H, t), 2.74 (2H, t). MS: APCI(+ve) 425/427 (M+H⁺).

### EXAMPLE 15

### (βR)-N-[6-Chloro-2-[(3S)-3-[(3-hydroxypropyl)amino]-1-pyrrolidinyl]-5-quinolinyl]-β-methyl-benzenepropanamide

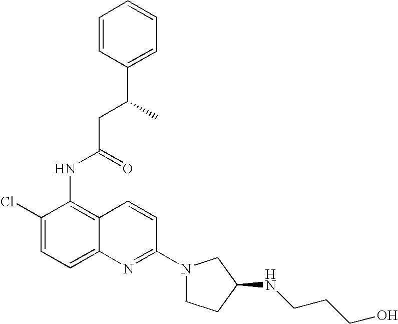

### (a) (βR)-N-[6-Chloro-2-[(3R)-3-hydroxy-1-pyrrolidinyl]-5-quinolinyl]-β-methyl-benzenepropanamide

To a 10 mL microwave vial was added (βR)-N-(2,6-dichloro-5-quinolinyl)-β-methyl-benzenepropanamide (Example 3(b)) (300 mg), (3R)-3-pyrrolidinol (220 mg) and acetonitrile (5 mL). The vial was sealed and heated at 100° C. for 45 minutes within a microwave. The reaction was cooled to room temperature and the resulting solid removed by filtration and washed with acetonitrile to afford the sub-title compound (340 mg).

¹H NMR (400 MHz, d₆-DMSO) δ 9.78 (1H, s), 7.51 (1H, d), 7.44 (1H, d), 7.40-7.31 (5H, m), 7.29-7.23 (1H, m), 6.74 (1H, d), 4.99 (1H, s), 4.41 (1H, s), 3.63-3.53 (2H, m), 3.39-3.22 (3H, m), 2.77 (1H, dd), 2.68 (1H, dd), 2.11-1.98 (1H, m), 1.97-1.88 (1H, m), 1.33 (3H, d).

### (b) (βR)-N-[6-Chloro-2-[(3R)-3-[(methylsulfonyl)oxy]-1-pyrrolidinyl]-5-quinolinyl]-β-methyl-benzenepropanamide

To a stirred solution of (βR)-N-[6-chloro-2-[(3R)-3-hydroxy-1-pyrrolidinyl]-5-quinolinyl]-β-methyl-benzenepropanamide (Example 15(a)) (340 mg) in dichloromethane was added methanesulphonyl chloride (0.26 mL) and triethylamine (0.46 mL). The reaction was stirred for 12 hours under nitrogen and then purified (SiO₂, methanol:dichloromethane:ammonium hydroxide solution 10:90:1 as eluant) to afford the sub-titled compound (250 mg).

¹H NMR (400 MHz, d₆-DMSO) δ 9.80 (1H, s), 7.55 (1H, d) 7.48 (1H, d), 7.44-7.32 (5H, m), 7.30-7.23 (1H, m), 6.81 (1H, d), 5.45 (1H, s), 3.93-3.69 (3H, m), 3.64-3.51 (1H, m), 3.35-3.29 (1H, m), 3.27 (3H, s), 2.78 (1H, dd), 2.68 (1H, dd), 2.38-2.28 (2H, m), 1.33 (3H, d).

### (c) (βR)-N-[6-Chloro-2-[(3S)-3-[(3-hydroxypropyl)amino]-1-pyrrolidinyl]-5-quinolinyl]-β-methyl-benzenepropanamide

To a 10 mL vial was added (βR)-N-[6-chloro-2-[(3R)-3-[(methylsulfonyl)oxy]-1-pyrrolidinyl]-5-quinolinyl]-β-methyl-benzenepropanamide (Example 15(b)) (130 mg), 3-amino-1-propanol (0.5 mL) and acetonitrile (3 mL). The vial was sealed and heated at 100° C. for 90 minutes within a microwave. The reaction was cooled to room temperature and evaporated. Purification (SiO₂, methanol:dichloromethane 1:9 as eluant) and recrystallisation (acetonitrile) afforded the title compound as a solid (21 mg).

¹H NMR (400 MHz, CD₃OD) δ 7.47 (1H, d), 7.41 (1H, d), 7.30-7.24 (4H, m), 7.23-7.16 (1H, m), 7.02 (1H, d), 6.56 (1H, d), 3.78-3.71 (1H, m), 3.68-3.61 (1H, m), 3.56 (2H, t), 3.51-3.35 (2H, m), 3.33-3.24 (2H, m), 2.82-2.73 (1H, m), 2.71-2.64 (3H, m), 2.25-2.14 (1H, m), 1.90-1.77 (1H, m), 1.67 (2H, dt), 1.32 (3H, d). MS: APCI(+ve) 467/469 (M+H⁺). m.p. 155-158° C.

### EXAMPLE 16

### (βR)-N-[6-Chloro-2-[(3S)-3-[(2-hydroxyethyl)amino]-1-pyrrolidinyl]-5-quinolinyl]-β-methyl-benzenepropanamide, dihydrochloride

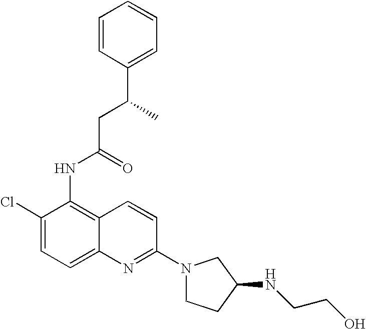

### a) (βR)-N-[6-Chloro-2-[(3S)-3-[[2-[[(1,1-dimethylethyl)dimethylsilyl]oxy]ethyl]amino]-1-pyrrolidinyl]-5-quinolinyl]-β-methyl-benzenepropanamide

A suspension of N-[2-[(3S)-3-amino-1-pyrrolidinyl]-6-chloro-5-quinolinyl]-β-methyl-, (βR)-benzenepropanamide (Example 13) (400 mg) and activated 3A molecular sieves (500 mg) in methanol (10 mL) was treated with (tert-butyldimethylsilyloxy)acetaldehyde (0.17 mL) and the resulting mixture stirred at room temperature for 6 hours. Sodium triacetoxyborohydride (416 mg) was added and the mixture stirred for 16 hours. The reaction mixture was concentrated to dryness. Purification (SiO₂, ethyl acetate:isohexane 1:1 as eluant) gave the sub-title compound as a solid (250 mg).

¹H NMR (400 MHz, CD₃OD) δ 7.52 (1H, d), 7.46 (1H, d), 7.35-7.19 (6H, m), 7.06 (1H, d), 6.61 (1H, d), 3.84-3.63 (4H, m), 3.59-3.48 (2H, m), 3.43-3.28 (2H, m), 2.87-2.64 (4H, m), 2.33-2.20 (1H, m), 1.97-1.84 (1H, m), 1.37 (3H, d), 0.85 (9H, s), 0.04 (6H, s).

### b) (βR)-N-[6-Chloro-2-[(3S)-3-[(2-hydroxyethyl)amino]-1-pyrrolidinyl]-5-quinolinyl]-β-methyl-benzenepropanamide, dihydrochloride

Trifluoroacetic acid (2 mL) was added to a stirred solution of (βR)-N-[6-chloro-2-[(3S)-3-[[2-[[(1,1-dimethylethyl)dimethylsilyl]oxy]ethyl]amino]-1-pyrrolidinyl]-5-quinolinyl]-β-methyl-benzenepropanamide (Example 16(a)) (250 mg) in dichloromethane (5 mL). The mixture was stirred at room temperature for 5 hours then concentrated. Purification (SiO₂, dichloromethane:methanol:7N ammonia in methanol 97:2:1 as eluant) and further purification (Varian SCX cartridge using methanol (100 mL) and then 7N ammonia in methanol (100 mL) as eluant) gave the title compound as a solid (40 mg).

¹H NMR (400 MHz, CD₃OD) δ 7.47 (1H, d), 7.41 (1H, d), 7.31-7.24 (4H, m), 7.20 (1H, quintet), 7.02 (1H, d), 6.56 (1H, d), 3.75 (1H, dd), 3.69-3.56 (3H, m), 3.52-3.42 (2H, m), 3.37-3.24 (2H, m), 2.83-2.63 (4H, m), 2.28-2.15 (1H, m), 1.94-1.80 (1H, m), 1.32 (3H, d). MS: APCI(+ve) 453.2/455.2 (M+H⁺). m.p. 177-182° C.

### EXAMPLE 17

### N-[6-Chloro-2-(1-piperazinyl)-5-quinolinyl]-benzenepropanamide

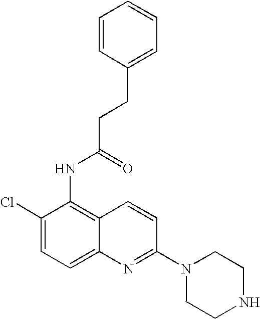

Prepared according to the method of Example 10(b) using 4-(5-amino-6-chloro-2-quinolinyl)-1-piperazinecarboxylic acid, 1,1-dimethylethyl ester (Example 10(a)) (200 mg) and benzenepropanoic acid (166 mg). Purification (SiO₂, dichloromethane:methanol:7N ammonia in methanol 90:10:1 as eluant) and recrystallisation from acetonitrile gave the title compound as a solid (17 mg).

¹H NMR (400 MHz, d₆-DMSO) δ 9.86 (1H, s), 7.66-7.55 (2H, m), 7.49 (1H, d), 7.38-7.28 (4H, m), 7.28-7.22 (1H, m), 7.18 (1H, d), 3.75-3.66 (4H, m), 3.03-2.89 (6H, m), 2.82-2.72 (2H, m). MS: APCI(+ve) 395/397 (M+H⁺). m.p. 231-234° C.

### EXAMPLE 18

### N-[2-[(3S)-3-Amino-1-pyrrolidinyl]-6-chloro-5-quinolinyl]-2-chlorobenzenepropanamide

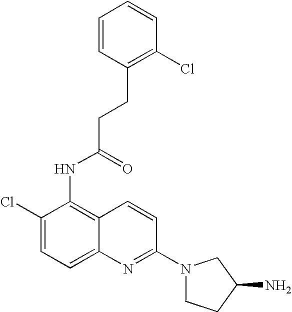

### a) 2-Chloro-N-(2,6-dichloro-5-quinolinyl)-benzenepropanamide

To a stirred solution of 2-chloro-benzenepropanoic acid (1 g) in dichloromethane (5 mL) at 0° C. under nitrogen, was added N,N-dimethylformamide (1 drop) and oxalyl chloride (2.4 mL). The reaction mixture was stirred at room temperature for 2 hours, then evaporated to dryness and redissolved in dichloromethane (2 mL). The solution was added to a mixture of 2,6-dichloroquinoline-5-amine (prepared as described in WO2003080579) (400 mg) and potassium carbonate (522 mg) in acetone (10 mL). The reaction mixture was stirred at room temperature for 2 hours. The resulting solid was collected by filtration and subsequently washed with water (10 mL) to give the sub-title compound (420 mg).

¹H NMR (400 MHz, d₆-DMSO) δ 10.19 (1H, s), 8.08 (1H, d), 7.93 (2H, s), 7.63 (1H, d), 7.52-7.40 (2H, m), 7.37-7.27 (2H, m), 3.09 (2H, t), 2.85 (2H, t). MS: APCI(+ve) 379 (M+H⁺).

### b) N-[2-[(3S)-3-Amino-1-pyrrolidinyl]-6-chloro-5-quinolinyl]-2-chlorobenzenepropanamide

Prepared according to the method of Example 13 using 2-chloro-N-(2,6-dichloro-5-quinolinyl)-benzenepropanamide (Example 18(a)) (420 mg) and (3S)-3-pyrrolidinamine (287 mg). Purification (SiO₂, dichloromethane:methanol:7N ammonia in methanol 90:10:1 as eluant) gave the title compound as a solid (335 mg).

¹H NMR (400 MHz, CD₃OD) δ 7.58-7.39 (3H, m), 7.37-7.26 (2H, m), 7.22-7.13 (2H, m), 6.71 (1H, d), 3.74-3.62 (2H, m), 3.62-3.47 (2H, m), 3.26 (1H, dd), 3.11 (2H, t), 2.80 (2H, t), 2.24-2.10 (1H, m), 1.87-1.73 (1H, m). MS: APCI(+ve) 429/431 (M+H⁺). m.p. 200-212° C.

### EXAMPLE 19

### 2-Chloro-N-[6-chloro-2-[(3S)-3-[(2-hydroxyethyl)amino]-1-pyrrolidinyl]-5-quinolinyl]-benzenepropanamide

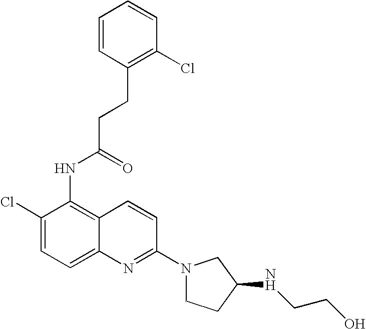

### a) 2-Chloro-N-[6-chloro-2-[(3S)-3-[[2-[[(1,1-dimethylethyl)dimethylsilyl]oxy]ethyl]amino]-1-pyrrolidinyl]-5-quinolinyl]-benzenepropanamide

Prepared according to the method of Example 16(a) using N-[2-[(3S)-3-amino-1-pyrrolidinyl]-6-chloro-5-quinolinyl]-2-chloro-benzenepropanamide (Example 18) (300 mg) and (tert-butyldimethylsilyloxy)acetaldehyde (0.12 mL). Purification (SiO₂, Ethyl acetate:isohexane 2:1 as eluant) gave the sub-title compound (200 mg).

¹H NMR (400 MHz, CD₃OD) δ 7.56-7.50 (2H, m), 7.45 (1H, d), 7.36-7.27 (2H, m), 7.21-7.14 (2H, m), 6.73 (1H, d), 3.81-3.62 (4H, m), 3.56-3.45 (2H, m), 3.41-3.35 (1H, m), 3.12 (2H, t), 2.85-2.72 (4H, m), 2.29-2.19 (1H, m), 1.92-1.83 (1H, m), 0.81 (9H, s), 0.01 (6H, s).

### b) 2-Chloro-N-[6-chloro-2-[(3S)-3-[(2-hydroxyethyl)amino]-1-pyrrolidinyl]-5-quinolinyl]-benzenepropanamide

Hydrochloric acid (2 mL, 4 M solution in 1,4-dioxane) was added to a stirred solution of 2-chloro-N-[6-chloro-2-[(3S)-3-[[2-[[(1,1-dimethylethyl)dimethylsilyl]oxy]ethyl]amino]-1-pyrrolidinyl]-5-quinolinyl]-benzenepropanamide (Example 19(a)) (200 mg). The mixture was stirred at room temperature under nitrogen for 45 minutes then concentrated. Purification (SiO₂, dichloromethane:methanol:7N ammonia in methanol 93:7:1 as eluant) gave the title compound as a solid (77 mg).

¹H NMR (400 MHz, d₆-DMSO) δ 9.86 (1H, s), 7.67 (1H, d), 7.54 (1H, d), 7.50-7.39 (3H, m), 7.37-7.25 (2H, m), 6.85 (1H, d), 4.49 (1H, t), 3.75-3.25 (6H, m), 3.08 (2H, t), 2.79 (2H, t), 2.65 (2H, t), 2.19-2.05 (1H, m), 1.92-1.75 (1H, m). MS: APCI(+ve) 473/475 (M+H⁺). m.p. 180-182° C.

### EXAMPLE 20

### 1-[6-Chloro-5-[[3-(2-chlorophenyl)-1-oxopropyl]amino]-2-quinolinyl]-4-piperidinecarboxylic acid, potassium salt

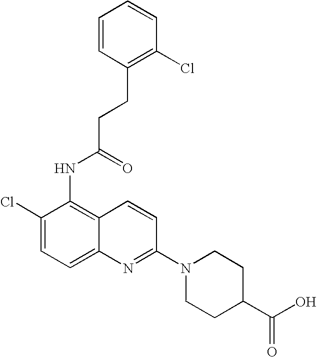

### a) 1-(5-Amino-6-chloro-2-quinolinyl)-4-piperidinecarboxylic acid ethyl ester

Prepared according to the method of Example 13 using 2,6-dichloro-5-quinolinamine (prepared as described in WO2003080579) (800 mg) and 4-piperidinecarboxylic acid, ethyl ester (1.8 g). Purification (SiO₂, dichloromethane:methanol 99:1 as eluant) gave sub-title compound as a solid (900 mg).

¹H NMR (400 MHz, d₆-DMSO) δ 8.34 (1H, d), 7.29 (1H, d), 7.14 (1H, d), 6.78 (1H, d), 5.84 (2H, s), 4.40 (2H, d), 4.07 (2H, q), 3.03 (2H, t), 2.69-2.58 (1H, m), 1.90 (2H, d), 1.55 (2H, q), 1.19 (3H, t). MS: APCI(+ve) 334/336 (M+H⁺).

### b) 1-[6-Chloro-5-[[3-(2-chlorophenyl)-1-oxopropyl]amino]-2-quinolinyl]-4-piperidinecarboxylic acid, ethyl ester

Prepared according to the method of Example 18 (a) using 1-(5-amino-6-chloro-2-quinolinyl)-4-piperidinecarboxylic acid ethyl ester (Example 20(a)) (200 mg) and 2-chloro-benzenepropanoic acid (330 mg). Solid product was collected by filtration and washed with water to give the sub-title compound (230 mg).

¹H NMR (400 MHz, CD₃OD) δ 9.91 (1H, s), 7.71 (1H, d), 7.58 (1H, d), 7.53-7.40 (3H, m), 7.38-7.21 (3H, m), 4.43 (2H, d), 4.08 (2H, q), 3.17-3.03 (4H, m), 2.80 (2H, t), 2.72-2.62 (1H, m), 1.93 (2H, d), 1.57 (2H, q), 1.19 (3H, t). MS: APCI(+ve) 500/502 (M+H⁺).

### c) 1-[6-Chloro-5-[[3-(2-chlorophenyl)-1-oxopropyl]amino]-2-quinolinyl]-4-piperidinecarboxylic acid, potassium salt

Potassium hydroxide (100 mg), in water (1 mL) was added to a solution of 1-[6-chloro-5-[[3-(2-chlorophenyl)-1-oxopropyl]amino]-2-quinolinyl]-4-piperidinecarboxylic acid, ethyl ester (Example 20(b)) (230 mg) in methanol (2 mL), in a 10 mL vial. The vial was sealed and heated at 70° C. for 10 minutes within a microwave. The reaction mixture was concentrated and water (10 mL) was added to the residue. The solid was collected by filtration to give the title compound (160 mg).

¹H NMR (300 MHz, d₆-DMSO) δ 7.73 (1H, d), 7.53-7.38 (4H, m), 7.32-7.20 (2H, m), 7.10 (1H, d), 4.27-4.13 (2H, m), 3.22-2.91 (4H, m), 2.82-2.68 (2H, m), 2.06-1.95 (1H, m), 1.84-1.71 (2H, m), 1.66-1.49 (2H, m). MS: APCI(+ve) 472/474 (M+H⁺).

### EXAMPLE 21

### 2-[(3S)-3-Amino-1-pyrrolidinyl]-6-chloro-N-[2-(2-chlorophenyl)ethyl]-5-quinolinecarboxamide

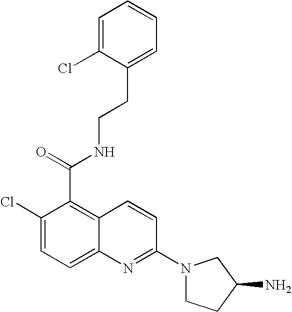

### a) 6-Chloro-N-[2-(2-chlorophenyl)ethyl]-5-quinolinecarboxamide

To a solution of 5-bromo-6-chloroquinoline (prepared according to the method of Journal of Heterocyclic Chemistry 1967, 4, 410) (3 g), 2-chloro-benzeneethanamine (3.8 g) and triethylamine (1.9 mL) in N-methyl pyrrolidinone (12 mL) was added dichlorobis(triphenylphosphine)palladium (II) (1.2 g). The mixture was heated with stirring at 100° C. under a 6 bar pressure of carbon monoxide for 16 hours after which it was cooled and filtered through diatamaceous earth, washing with methanol. The combined organics were concentrated and the residue was partitioned between dichloromethane (100 mL) and water (100 mL). The layers were separated and the aqueous was extracted with dichloromethane (2×100 mL). The combined organics were washed with 2M aqueous hydrochloric acid (50 mL) and saturated aqueous sodium hydrogen carbonate (50 mL) and then dried, filtered and evaporated. Purification (SiO₂, dichloromethane:methanol 95:5 as eluant) gave the sub-title compound as a solid (2 g).

¹H NMR (400 MHz, d₆-DMSO) δ 9.00-8.86 (2H, m), 8.06 (1H, d), 7.92-7.77 (2H, m), 7.63-7.53 (1H, m), 7.52-7.38 (2H, m), 7.36-7.24 (2H, m), 3.77-3.60 (2H, m), 3.10-2.98 (2H, m).

### b) 6-Chloro-N-[2-(2-chlorophenyl)ethyl]-5-quinolinecarboxamide 1-oxide

To a stirred solution of 6-chloro-N-[2-(2-chlorophenyl)ethyl]-5-quinolinecarboxamide (Example 21(a)) (2 g) in acetic acid (20 mL) was added peracetic acid 36-40 wt. % solution in acetic acid (10 mL). The mixture was stirred at room temperature for 16 hours then added to a solution of 10% aqueous sodium sulfite (100 mL) which was subsequently extracted with dichloromethane (3×100 mL). The combined extracts were washed with saturated aqueous sodium hydrogen carbonate (2×50 mL), dried, filtered and evaporated. Purification (SiO₂, dichloromethane:methanol 98:2 as eluant) gave the sub-title compound as a solid (1 g).

¹H NMR (400 MHz, d₆-DMSO) δ 8.97 (1H, t), 8.63 (1H, d), 8.55 (1H, d), 7.87 (1H, d), 7.54-7.37 (4H, m), 7.35-7.27 (2H, m), 3.67 (2H, q), 3.04 (2H, t). MS: APCI(+ve) 361 (M+H⁺).

### c) 2,6-Dichloro-N-[2-(2-chlorophenyl)ethyl]-5-quinolinecarboxamide

Phosphorus oxychloride (6 mL) was added drop wise to a suspension of 6-chloro-N-[2-(2-chlorophenyl)ethyl]-5-quinolinecarboxamide 1-oxide (Example 21(b)) (1 g) in dichloromethane (3 mL) at 0° C. The reaction mixture was then heated to 60° C. for 2 hours then allowed to cool and concentrated. The residue was partitioned between dichloromethane (100 mL) and ice water (50 mL). The organic layer was separated and the aqueous layer was extracted with dichloromethane (3×50 mL). The combined organics were washed with saturated aqueous sodium hydrogen carbonate (50 mL), dried, filtered and evaporated. Purification (SiO₂, ethyl acetate:isohexane 1:3 as eluant) gave the sub-title compound (700 mg).

¹H NMR (400 MHz, d₆-DMSO) δ 8.94 (1H, t), 8.01 (1H, d), 7.90 (2H, t), 7.65 (1H, d), 7.50-7.40 (2H, m), 7.35-7.28 (2H, m), 3.67 (2H, q), 3.03 (2H, t). MS: APCI(+ve) 379/381 (M+H⁺).

### d) 2-[(3S)-3-Amino-1-pyrrolidinyl]-6-chloro-N-[2-(2-chlorophenyl)ethyl]-5-quinolinecarboxamide

Prepared according to the method of Example 13 using 2,6-dichloro-N-[2-(2-chlorophenyl)ethyl]-5-quinolinecarboxamide (500 mg) and (3S)-3-pyrrolidinamine (354 mg). Purification (SiO₂, dichloromethane:methanol:7N ammonia in methanol 95:5:1) gave the title compound as a solid (450 mg).

¹H NMR (400 MHz, d₆-DMSO) δ 8.77 (1H, t), 7.57-7.39 (5H, m), 7.35-7.27 (2H, m), 6.85 (1H, d), 3.72-3.47 (6H, m), 3.27-3.13 (1H, m), 3.01 (2H, t), 2.13-2.01 (1H, m), 1.80-1.64 (3H, m). MS: APCI(+ve) 429/431 (M+H⁺). m.p. 196-198° C.

### EXAMPLE 22

### 6-Chloro-N-[2-(2-chlorophenyl)ethyl]-2-[(3S)-3-[(2-hydroxyethyl)amino]-1-pyrrolidinyl]-5-quinolinecarboxamide

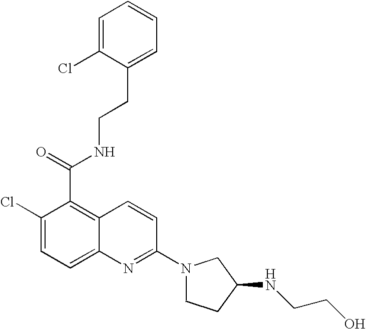

### a) 6-Chloro-N-[2-(2-chlorophenyl)ethyl]-2-[(3S)-3-[[2-[[(1,1-dimethylethyl)dimethylsilyl]oxy]ethyl]amino]-1-pyrrolidinyl]-5-quinolinecarboxamide

Prepared according to the method of Example 16(a) using 2-[(3S)-3-amino-1-pyrrolidinyl]-6-chloro-N-[2-(2-chlorophenyl)ethyl]-5-quinolinecarboxamide (Example 21) (300 mg) and (tert-butyldimethylsilyloxy)acetaldehyde (0.12 mL). Purification (SiO₂, dichloromethane:methanol 95:5 as eluant) gave the sub-title compound (320 mg).

¹H NMR (400 MHz, d₆-DMSO) δ 8.77 (1H, t), 7.56-7.39 (5H, m), 7.34-7.26 (2H, m), 6.87 (1H, d), 3.76-3.19 (9H, m), 3.01 (2H, t), 2.74-2.63 (2H, m), 2.18-2.05 (1H, m), 1.87-1.75 (1H, m), 0.86 (9H, s), 0.04 (6H, s).

### b) 6-Chloro-N-[2-(2-chlorophenyl)ethyl]-2-[(3S)-3-[(2-hydroxyethyl)amino]-1-pyrrolidinyl]-5-quinolinecarboxamide

Prepared according to the method of Example 19(b) using 6-chloro-N-[2-(2-chlorophenyl)ethyl]-2-[(3S)-3-[[2-[[(1,1-dimethylethyl)dimethylsilyl]oxy]ethyl]amino]-1-pyrrolidinyl]-5-quinolinecarboxamide (Example 22(a)) (320 mg). Purification by HPLC (Symmetry 0.1% aqueous trifluoroacetic acid/acetonitrile) gave the title compound as a solid (69 mg).

¹H NMR (300 MHz, d₆-DMSO) δ 8.77 (1H, t), 7.59-7.38 (5H, m), 7.36-7.25 (2H, m), 6.87 (1H, d), 4.51 (1H, s), 3.77-3.19 (7H, m), 3.01 (2H, t), 2.66 (2H, t), 2.20-2.05 (1H, m), 1.91-1.77 (1H, m). MS: APCI(+ve) 473/475 (M+H⁺). m.p. 170-172° C.

### EXAMPLE 23

### 1-[6-Chloro-5-[[[2-(2,6-dichlorophenyl)ethyl]amino]carbonyl]-2-quinolinyl]-4-piperidinecarboxylic acid

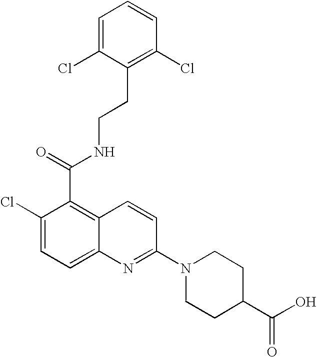

### a) 5-Bromo-2,6-dichloro-quinoline

2,6-Dichloroquinoline (30 g) and aluminium trichloride (60 g) were heated to 120° C. with stirring under a nitrogen atmosphere. Bromine (9.2 mL) was added dropwise over 1 hour and the mixture was then stirred at 120° C. for 1 hour before being cooled to room temperature. A methanol/deionised water mixture (150 mL, 1:1) was then slowly added and the mixture was concentrated in vacuo. Dichloromethane (500 mL) and deionised water (250 mL) were added, the layers were separated and the aqueous fraction was extracted with dichloromethane (2×250 mL). The combined organic extracts were washed with saturated aqueous sodium hydrogen carbonate (250 mL) before being dried, filtered and concentrated. Purification by chromatography (SiO₂, isohexane:dichloromethane 7:3 as eluant) gave the sub-title compound as a solid (27 g).

¹H NMR (400 MHz, CDCl₃) δ 8.53 (1H, d), 7.94 (1H, d), 7.78 (1H, d), 7.50 (1H, d). MS: APCI(+ve) 276/278/280/282 (M+H⁺).

### b) 2,6-Dichloro-5-quinolinecarboxylic acid

To a stirred solution of 5-bromo-2,6-dichloro-quinoline (23 g) in tetrahydrofuran (300 mL) at 0° C. was added iso-propylmagnesium chloride (2M in tetrahydrofuran, 42 mL) over 2 hours. CO₂ was bubbled through the solution for 20 minutes and then methanol (20 mL) was added. The mixture was poured into water (500 mL) and extracted with ethyl acetate. The aqueous layer was acidified with hydrochloric acid (2M in water) to pH2-3 and the resulting solid collected by filtration. The solid was washed with water and dried to afford the sub-titled compound (11.5 g).

¹H NMR (400 MHz, d₆-DMSO) δ 8.29 (1H, d), 8.07 (1H, d), 7.94 (1H, d), 7.74 (1H, d).

### c) 6-Chloro-2-[4-(ethoxycarbonyl)-1-piperidinyl]-5-quinolinecarboxylic acid

Prepared according to the method of Example 13 using 2,6-dichloro-5-quinolinecarboxylic acid (Example 23(b)) (800 mg) and 4-piperidinecarboxylic acid, ethyl ester (2.7 g). Purification (SiO₂, dichloromethane:methanol 99:1 as eluant) and further purification (Varian NH₂ cartridge using methanol (100 mL) and then 5% acetic acid in methanol (100 mL) as eluant) gave sub-title compound as a solid (900 mg).

¹H NMR (400 MHz, d₆-DMSO) δ 7.85 (1H, d), 7.62-7.53 (2H, m), 7.38 (1H, d), 4.43 (2H, d), 4.08 (2H, q), 3.11 (2H, t), 2.72-2.60 (1H, m), 1.97-1.87 (2H, m), 1.56 (2H, q), 1.19 (3H, t). MS: APCI(+ve) 363/365 (M+H⁺).

### d) 1-[6-Chloro-5-[[[2-(2,6-dichlorophenyl)ethyl]amino]carbonyl]-2-quinolinyl]-4-piperidinecarboxylic acid ethyl ester

Prepared according to the method of Example 1(b) using 6-chloro-2-[4-(ethoxycarbonyl)-1-piperidinyl]-5-quinolinecarboxylic acid (Example 23(c)) (220 mg) and 2,6-dichlorobenzenepropanoic acid (323 mg). Purification (SiO₂, dichloromethane:methanol 99:1 as eluant) gave the sub-title compound (240 mg).

¹H NMR (400 MHz, d₆-DMSO) δ 8.87 (1H, t), 7.67 (1H, d), 7.58-7.48 (4H, m), 7.36-7.30 (2H, m), 4.43 (2H, d), 4.08 (2H, q), 3.56 (2H, q), 3.21 (2H, t), 3.11 (2H, t), 2.73-2.60 (1H, m), 1.93 (2H, d), 1.56 (2H, q), 1.19 (3H, t). MS: APCI(+ve) 534/536 (M+H⁺).

### e) 1-[6-Chloro-5-[[[2-(2,6-dichlorophenyl)ethyl]amino]carbonyl]-2-quinolinyl]-4-piperidinecarboxylic acid

Prepared according to the method of Example 20(c) using 1-[6-chloro-5-[[[2-(2,6-dichlorophenyl)ethyl]amino]carbonyl]-2-quinolinyl]-4-piperidinecarboxylic acid ethyl ester (Example 23(b)) (240 mg). The reaction mixture was acidified to pH5 using 2 M aqueous hydrochloric acid and the solid was collected by filtration. Purification (Varian NH₂ cartridge using methanol (100 mL) and then 5% acetic acid in methanol (100 mL) as eluant) gave the title compound as a solid (115 mg).

¹H NMR (300 MHz, d₆-DMSO) δ 8.92-8.80 (1H, m), 7.66 (1H, d), 7.57-7.44 (4H, m), 7.38-7.28 (2H, m), 4.42 (2H, d), 3.66-3.46 (2H, m), 3.27-2.97 (5H, m), 2.01-1.81 (2H, m), 1.64-1.45 (2H, m). MS: APCI(+ve) 506 (M+H⁺). m.p. 262-264° C.

### EXAMPLE 24

### 1-[6-Chloro-5-[[[2-(2-chlorophenyl)ethyl]amino]carbonyl]-2-quinolinyl]-4-piperidinecarboxylic acid

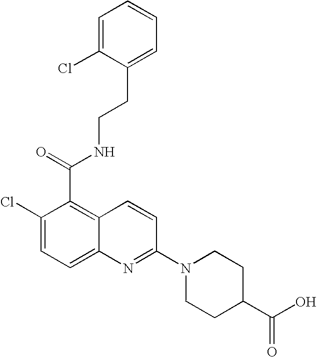

### a) 1-[6-Chloro-5-[[[2-(2-chlorophenyl)ethyl]amino]carbonyl]-2-quinolinyl]-4-piperidinecarboxylic acid ethyl ester

Prepared according to the method of Example 1 using 6-chloro-2-[4-(ethoxycarbonyl)-1-piperidinyl]-5-quinolinecarboxylic acid (Example 23(c)) (220 mg) and 2-chlorobenzeneethanamine (265 mg). Purification (SiO₂, dichloromethane:methanol 99:1 as eluant) gave the sub-title compound (160 mg).

¹H NMR (400 MHz, d₆-DMSO) δ 8.77 (1H, t), 7.60-7.39 (5H, m), 7.35-7.24 (3H, m), 4.42 (2H, d), 4.08 (2H, q), 3.63 (2H, q), 3.10 (2H, t), 3.01 (2H, t), 2.73-2.62 (1H, m), 1.92 (2H, d), 1.55 (2H, q), 1.19 (3H, t). MS: APCI(+ve) 500/502 (M+H⁺).

### b) 1-[6-Chloro-5-[[[2-(2-chlorophenyl)ethyl]amino]carbonyl]-2-quinolinyl]-4-piperidinecarboxylic acid

Prepared according to the method of Example 20(c) using 1-[6-chloro-5-[[[2-(2-chlorophenyl)ethyl]amino]carbonyl]-2-quinolinyl]-4-piperidinecarboxylic acid ethyl ester (Example 24(a)) (160 mg). Reaction mixture was acidified to pH5 using 2 M aqueous hydrochloric acid and the solid was collected by filtration. Purification (Varian NH₂ cartridge using methanol:dichloromethane 1:1 (100 mL) and then acetic acid:methanol:dichloromethane 1:10:10 (100 mL) as eluant) gave the title compound as a solid (70 mg).

¹H NMR (300 MHz, d₆-DMSO) δ 8.76 (1H, t), 7.61-7.38 (5H, m), 7.37-7.23 (3H, m), 4.41 (2H, d), 3.63 (2H, q), 3.16-2.96 (4H, m), 2.63-2.39 (1H, m), 1.95-1.84 (2H, m), 1.65-1.43 (2H, m). MS: APCI(−ve) 470/472 (M−H⁺). m.p. 250-253° C.

### EXAMPLE 25

### 1-[6-Chloro-5-[[(2,2-diphenylethyl)amino]carbonyl]-2-quinolinyl]-4-piperidinecarboxylic acid, acetate

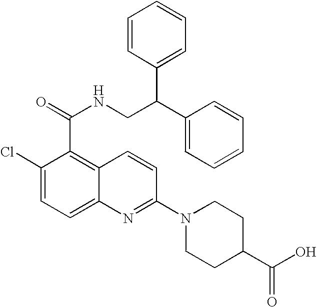

### a) 1-[6-Chloro-5-[[(2,2-diphenylethyl)amino]carbonyl]-2-quinolinyl]-4-piperidinecarboxylic acid ethyl ester

Prepared according to the method of Example 1 using 6-chloro-2-[4-(ethoxycarbonyl)-1-piperidinyl]-5-quinolinecarboxylic acid (Example 23(c)) (220 mg) and β-phenylbenzeneethanamine (335 mg). Purification (SiO₂, dichloromethane as eluant) gave the sub-title compound (250 mg).

¹H NMR (300 MHz, d₆-DMSO) δ 8.78-8.68 (1H, m), 7.55-6.95 (14H, m), 4.45-4.30 (3H, m), 4.14-3.96 (4H, m), 3.20-2.98 (2H, m), 2.76-2.59 (1H, m), 2.01-1.81 (2H, m), 1.54 (2H, q), 1.19 (3H, t). MS: APCI(+ve) 542/544 (M+H⁺).

### b) 1-[6-Chloro-5-[[(2,2-diphenylethyl)amino]carbonyl]-2-quinolinyl]-4-piperidinecarboxylic acid, acetate

Prepared according to the method of Example 20(c) using 1-[6-chloro-5-[[(2,2-diphenylethyl)amino]carbonyl]-2-quinolinyl]-4-piperidinecarboxylic acid ethyl ester (Example 25(a)) (250 mg). Reaction mixture was acidified to pH 5 using 2M aqueous hydrochloric acid and the solid was collected by filtration. Purification (Varian NH₂ cartridge using methanol (100 mL) and then 5% acetic acid in methanol (100 mL) as eluant) gave the title compound as a solid (160 mg).

¹H NMR (300 MHz, d₆-DMSO) δ 8.73 (1H, t), 7.53-7.19 (12H, m), 7.10 (1H, d), 6.99 (1H, d), 4.46-4.27 (3H, m), 4.01 (2H, t), 3.04 (2H, t), 2.59-2.33 (1H, m), 1.98-1.74 (2H, m), 1.62-1.40 (2H, m). MS: APCI(−ve) 512/514 (M−H⁺). m.p. 180-185° C.

### EXAMPLE 26

### 1-[6-Chloro-5-[[(2-phenylethyl)amino]carbonyl]-2-quinolinyl]-4-piperidinecarboxylic acid

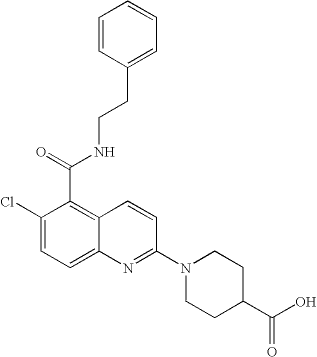

### a) 1-[6-Chloro-5-[[(2-phenylethyl)amino]carbonyl]-2-quinolinyl]-4-piperidinecarboxylic acid ethyl ester

Prepared according to the method of Example 1 using 6-chloro-2-[4-(ethoxycarbonyl)-1-piperidinyl]-5-quinolinecarboxylic acid (Example 23(c)) (220 mg) and benzeneethanamine (175 mg). The resulting solid was recrystallised from acetonitrile to give the sub-title compound (200 mg).

¹H NMR (400 MHz, d₆-DMSO) δ 8.71 (1H, t), 7.57-7.47 (3H, m), 7.37-7.19 (6H, m), 4.41 (2H, d), 4.08 (2H, q), 3.60 (2H, q), 3.10 (2H, t), 2.88 (2H, t), 2.72-2.62 (1H, m), 1.93 (2H, d), 1.55 (2H, q), 1.19 (3H, t). MS: APCI(+ve) 466/468 (M+H⁺).

### b) 1-[6-Chloro-5-[[(2-phenylethyl)amino]carbonyl]-2-quinolinyl]-4-piperidinecarboxylic acid

Prepared according to the method of Example 20(c) using 1-[6-chloro-5-[[(2-phenylethyl)amino]carbonyl]-2-quinolinyl]-4-piperidinecarboxylic acid ethyl ester (Example 26(a)) (200 mg). The reaction mixture was acidified to pH 5 using 2M aqueous hydrochloric acid and the solid was collected by filtration and washed with water to give the title compound (110 mg).

¹H NMR (400 MHz, d₆-DMSO) δ 12.26 (1H, s), 8.72 (1H, t), 7.59-7.46 (3H, m), 7.36-7.20 (6H, m), 4.41 (2H, d), 3.60 (2H, q), 3.11 (2H, t), 2.88 (2H, t), 2.62-2.53 (1H, m), 1.92 (2H, d), 1.55 (2H, q). MS: APCI(−ve) 436/438 (M−H⁺). m.p. 260-262° C.

### EXAMPLE 27

### 1-[6-Chloro-5-[[[2-(2-fluorophenyl)ethyl]amino]carbonyl]-2-quinolinyl]-4-piperidinecarboxylic acid

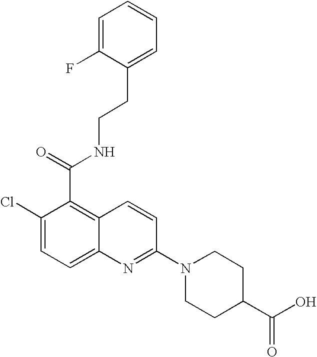

### a) 1-[6-Chloro-5-[[[2-(2-fluorophenyl)ethyl]amino]carbonyl]-2-quinolinyl]-4-piperidinecarboxylic acid ethyl ester

Prepared according to the method of Example 1(b) using 6-chloro-2-[4-(ethoxycarbonyl)-1-piperidinyl]-5-quinolinecarboxylic acid (Example 23(c)) (220 mg) and 2-fluorobenzeneethanamine (216 mg). The resulting solid was recrystallised from acetonitrile to give the sub-title compound (260 mg).

¹H NMR (400 MHz, d₆-DMSO) δ 8.75 (1H, t), 7.57-7.49 (3H, m), 7.41-7.14 (5H, m), 4.42 (2H, d), 4.08 (2H, q), 3.61 (2H, q), 3.10 (2H, t), 2.92 (2H, t), 2.67 (1H, tt), 1.92 (2H, d), 1.55 (2H, q), 1.19 (3H, t). MS: APCI(+ve) 484/486 (M+H⁺).

### b) 1-[6-Chloro-5-[[[2-(2-fluorophenyl)ethyl]amino]carbonyl]-2-quinolinyl]-4-piperidinecarboxylic acid

Prepared according to the method of Example 20(c) using 1-[6-chloro-5-[[[2-(2-fluorophenyl)ethyl]amino]carbonyl]-2-quinolinyl]-4-piperidinecarboxylic acid ethyl ester (Example 27(a)) (260 mg). The reaction mixture was acidified to pH 5 using 2M aqueous hydrochloric acid and the solid was collected by filtration and washed with water to give the title compound (125 mg).

¹H NMR (400 MHz, d₆-DMSO) δ 8.75 (1H, t), 7.57-7.49 (3H, m), 7.41-7.14 (5H, m), 4.41 (2H, d), 3.60 (2H, q), 3.09 (2H, t), 2.92 (2H, t), 2.61-2.52 (1H, m), 1.92 (2H, d), 1.53 (2H, q). MS: APCI(−ve) 454/456 (M−H⁺). m.p. 270-272° C.

### EXAMPLE 28

### 1-[6-Chloro-5-[[[2-(2-methylphenyl)ethyl]amino]carbonyl]-2-quinolinyl]-4-piperidinecarboxylic acid

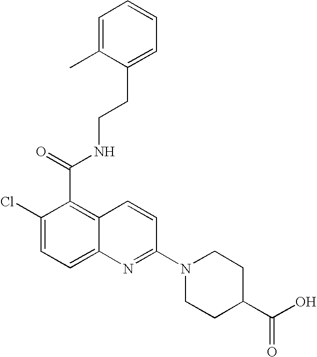

### a) 1-[6-Chloro-5-[[[2-(2-methylphenyl)ethyl]amino]carbonyl]-2-quinolinyl]-4-piperidinecarboxylic acid ethyl ester

Prepared according to the method of Example 1 using 6-chloro-2-[4-(ethoxycarbonyl)-1-piperidinyl]-5-quinolinecarboxylic acid (Example 23(c)) (220 mg) and 2-methyl-benzeneethanamine (164 mg). The resulting solid was recrystallised from acetonitrile to give the sub-title compound (180 mg).

¹H NMR (400 MHz, d₆-DMSO) δ 8.76 (1H, t), 7.60-7.51 (3H, m), 7.29-7.13 (5H, m), 4.42 (2H, d), 4.08 (2H, q), 3.54 (2H, q), 3.10 (2H, t), 2.88 (2H, t), 2.73-2.62 (1H, m), 2.35 (3H, s), 1.93 (2H, d), 1.55 (2H, q), 1.19 (3H, t). MS: APCI(+ve) 480/482 (M+H⁺).

### b) 1-[6-Chloro-5-[[[2-(2-methylphenyl)ethyl]amino]carbonyl]-2-quinolinyl]-4-piperidinecarboxylic acid

Prepared according to the method of Example 20(c) using 1-[6-chloro-5-[[[2-(2-methylphenyl)ethyl]amino]carbonyl]-2-quinolinyl]-4-piperidinecarboxylic acid ethyl ester (Example 28(a)) (180 mg). The reaction mixture was acidified to pH 5 using 2M aqueous hydrochloric acid and the solid was collected by filtration and washed with water to give the title compound (120 mg).

¹H NMR (400 MHz, d₆-DMSO) δ 8.88 (1H, s), 8.04-7.83 (1H, m), 7.68 (2H, d), 7.44 (1H, s), 7.26-7.10 (4H, m), 4.43 (2H, d), 3.55 (2H, q), 3.41-3.22 (2H, m), 2.89 (2H, t), 2.72-2.60 (1H, m), 2.35 (3H, s), 1.99 (2H, d), 1.65 (2H, d). MS: APCI(−ve) 450/452 (M−H⁺). m.p. 237-241° C.

### EXAMPLE 29

### 1-[6-Chloro-5-[[[(2S)-2-phenylpropyl]amino]carbonyl]-2-quinolinyl]-4-piperidinecarboxylic acid

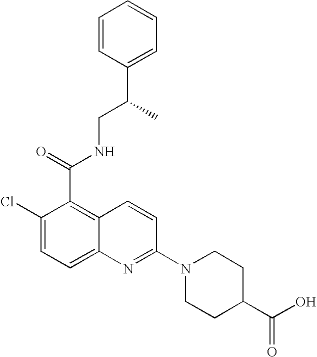

### a) 1-[6-Chloro-5-[[[(2S)-2-phenylpropyl]amino]carbonyl]-2-quinolinyl]-4-piperidinecarboxylic acid ethyl ester

Prepared according to the method of Example 1(b) using 6-chloro-2-[4-(ethoxycarbonyl)-1-piperidinyl]-5-quinolinecarboxylic acid (Example 23(c)) (220 mg) and (PS)-β-methyl-benzeneethanamine (150 mg). The resulting solid was recrystallised from acetonitrile to give the sub-title compound (230 mg).

¹H NMR (400 MHz, d₆-DMSO) δ 8.67 (1H, t), 7.55-7.47 (2H, m), 7.38-7.23 (6H, m), 7.17 (1H, d), 4.40 (2H, d), 4.07 (2H, q), 3.65 (1H, dt), 3.39 (1H, ddd), 3.15-3.01 (3H, m), 2.71-2.62 (1H, m), 1.92 (2H, d), 1.54 (2H, q), 1.28 (3H, d), 1.18 (3H, t). MS: APCI(+ve) 480/482 (M+H⁺).

### b) 1-[6-Chloro-5-[[[(2S)-2-phenylpropyl]amino]carbonyl]-2-quinolinyl]-4-piperidinecarboxylic acid

Prepared according to the method of Example 20(c) using 1-[6-chloro-5-[[[(2S)-2-phenylpropyl]amino]carbonyl]-2-quinolinyl]-4-piperidinecarboxylic acid ethyl ester (Example 29 (a)) (230 mg). The reaction mixture was acidified to pH 5 using 2 M aqueous hydrochloric acid and the solid was collected by filtration and washed with water to give the title compound (160 mg).

¹H NMR (400 MHz, d₆-DMSO) δ 8.35 (1H, t), 7.58 (1H, d), 7.49 (2H, t), 7.35-7.27 (4H, m), 7.26-7.20 (1H, m), 7.16 (1H, d), 4.33 (2H, d), 3.68-3.59 (1H, m), 3.49-3.40 (1H, m), 3.25-3.06 (3H, m), 2.63-2.53 (1H, m), 1.94 (2H, d), 1.62 (2H, q), 1.30 (3H, d). MS: APCI(−ve) 450/452 (M−H⁺). m.p. 150-153° C.

### EXAMPLE 30

### 6-Chloro-N-[2-(2-chlorophenyl)ethyl]-2-[4-(1,5-dihydro-5-oxo-4H-1,2,4-triazol-4-yl)-1-piperidinyl]-5-quinolinecarboxamide

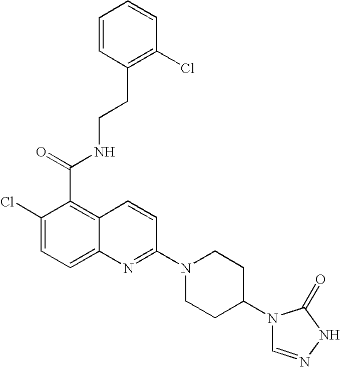

### a) 2-Formyl-N-[1-(phenylmethyl)-4-piperidinyl]-hydrazinecarboxamide

1-(Phenylmethyl)-4-piperidinamine (3 g) in dichloromethane (10 mL) and triethylamine (4.5 mL) were added dropwise to a stirred solution of triphosgene (1.55 g) in dichloromethane (20 mL) at 0° C. under nitrogen. The reaction mixture was allowed to warm to room temperature and stirred for 30 minutes. The mixture was cooled to 0° C. and formyl-hydrazine (1.4 g) and triethylamine (4.5 mL) were added. The reaction was stirred at room temperature for 1 hour, then evaporated to dryness. Purification (SiO₂, methanol:dichloromethane:ammonium hydroxide solution 5:95:1 as eluant) gave the sub-title compound (2.5 g).

MS: APCI(+ve) 277.2 (M+H⁺).

### b) 2,4-Dihydro-4-[1-(phenylmethyl)-4-piperidinyl]-3H-1,2,4-triazol-3-one

2-Formyl-N-[1-(phenylmethyl)-4-piperidinyl]-hydrazinecarboxamide (Example 30 (a)) (2.5 g) was divided between 5 10 mL vials. Potassium hydroxide (5 ml, 1 M solution in methanol) was added to each vial and the reactions were heated at 90° C. for 35 minutes within a microwave. The combined reaction mixtures were acidified to pH6 with aqueous 2M hydrochloric acid and then evaporated to dryness. Purification (SiO₂, methanol:dichloromethane:acetic acid 15:85:1 as eluant) gave the sub-title compound as an oil (2.2 g).

MS: APCI(+ve) 259.2 (M+H⁺).

### c) 2,4-Dihydro-4-(4-piperidinyl)-3H-1,2,4-triazol-3-one

2,4-Dihydro-4-[1-(phenylmethyl)-4-piperidinyl]-3H-1,2,4-triazol-3-one (Example 30(b)) (2.2 g) was divided between 2 10 mL vials. 1,4-Cyclohexadiene (5 mL) and palladium hydroxide (270 mg, 20 wt. % on carbon) were added to each vial and the reactions were heated at 100° C. for 30 minutes within a microwave. The reaction mixtures were combined. Ethanol (50 mL) and water (50 mL) were added and the mixture was filtered through diatomaceous earth and evaporated to give sub-title compound as a solid (720 mg).

MS: APCI(+ve) 169.2 (M+H⁺).

### d) 6-Chloro-N-[2-(2-chlorophenyl)ethyl]-2-[4-(1,5-dihydro-5-oxo-4H-1,2,4-triazol-4-yl)-1-piperidinyl]-5-quinolinecarboxamide

Prepared according to the method of Example 13, using 2,6-dichloro-N-[2-(2-chlorophenyl)ethyl]-5-quinolinecarboxamide (Example 21(c)) (150 mg) and 2,4-dihydro-4-(4-piperidinyl)-3H-1,2,4-triazol-3-one (Example 30(c)) (200 mg). Purification (SiO₂, methanol:dichloromethane 2:98 as eluant) gave the title compound as a solid (60 mg).

¹H NMR (400 MHz, d₆-DMSO) δ 11.65 (1H, s), 8.78 (1H, t), 7.97 (1H, s), 7.62-7.39 (5H, m), 7.35-7.26 (3H, m), 4.70 (2H, d), 4.13-4.01 (1H, m), 3.63 (2H, q), 3.12-2.96 (4H, m), 1.94 (2H, d), 1.79 (2H, q). MS: APCI(+ve) 511/513 (M+H⁺).

### EXAMPLE 31

### 1-[6-Chloro-5-[[[2-(4-chlorophenyl)ethyl]amino]carbonyl]-2-quinolinyl]-4-piperidinecarboxylic acid

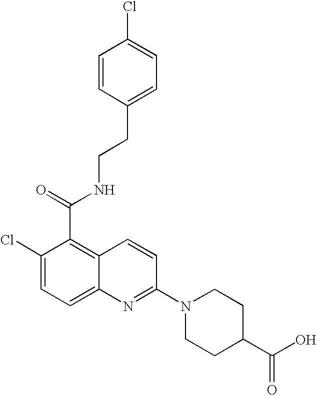

### a) 1-[6-Chloro-5-[[[2-(4-chlorophenyl)ethyl]amino]carbonyl]-2-quinolinyl]-4-piperidinecarboxylic acid ethyl ester

Prepared according to the method of Example 1 using 6-chloro-2-[4-(ethoxycarbonyl)-1-piperidinyl]-5-quinolinecarboxylic acid (Example 23 (c) (220 mg) and 4-chlorobenzeneethanamine (200 mg). The resulting solid was recrystallised from acetonitrile to give the sub-title compound (107 mg).

¹H NMR (400 MHz, d₆-DMSO) δ 8.68 (1H, t), 7.56-7.48 (2H, m), 7.43-7.29 (5H, m), 7.20 (1H, d), 4.41 (2H, d), 4.08 (2H, q), 3.60 (2H, q), 3.11 (2H, t), 2.88 (2H, t), 2.73-2.62 (1H, m), 1.92 (2H, d), 1.55 (2H, q), 1.19 (3H, t). MS: APCI(+ve) 502 (M+H⁺).

### b) 1-[6-Chloro-5-[[[2-(4-chlorophenyl)ethyl]amino]carbonyl]-2-quinolinyl]-4-piperidinecarboxylic acid

Prepared according to the method of Example 20 (c) using 1-[6-chloro-5-[[[2-(4-chlorophenyl)ethyl]amino]carbonyl]-2-quinolinyl]-4-piperidinecarboxylic acid ethyl ester (Example 31 (a)) (107 mg). The reaction mixture was acidified to pH 5 using 2 M aqueous hydrochloric acid and the solid was collected by filtration and washed with water to give the title compound (80 mg).

¹H NMR (400 MHz, d₆-DMSO) δ 8.45-8.36 (1H, m), 7.64 (1H, d), 7.57 (1H, d), 7.52 (1H, d), 7.37-7.26 (4H, m), 7.22 (1H, d), 4.34 (2H, d), 3.62 (2H, q), 3.23 (2H, t), 2.91 (2H, t), 2.65-2.54 (1H, m), 1.95 (2H, d), 1.64 (2H, q). MS: APCI(−ve) 470/472 (M−H⁺). m.p. 231-234° C.

### Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37(3), p. 126). Consequently, when the receptor is activated using bbATP in the presence of ethidium bromide (a fluorescent DNA probe), an increase in the fluorescence of intracellular DNA-bound ethidium bromide is observed. The increase in fluorescence can be used as a measure of P2X₇ receptor activation and therefore to quantify the effect of a compound on the P2X₇ receptor.

In this manner, each of the title compounds of the Examples was tested for antagonist activity at the P2X₇ receptor. Thus, the test was performed in 96-well flat bottomed microtitre plates, the wells being filled with 250 μl of test solution comprising 200 μl of a suspension of THP-1 cells (2.5×10⁶ cells/ml) containing 10⁻⁴M ethidium bromide, 25 μl of a high potassium buffer solution containing 10⁻⁵M bbATP, and 25 μl of the high potassium buffer solution containing 3×10⁻⁵M test compound. The plate was covered with a plastics sheet and incubated at 37° C. for one hour. The plate was then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) were used separately in the test as controls. From the readings obtained, a pIC₅₀ figure was calculated for each test compound, this figure being the negative logarithm of the concentration of test compound necessary to reduce the bbATP agonist activity by 50%. Each of the compounds of the Examples demonstrated antagonist activity, having a pIC₅₀ figure>5.5. For example, the following table shows the pIC₅₀ figures for a representative selection of compounds:

| Compound of | |
|---|---|
| Example No. | pIC₅₀ |
| 1 | 6.5 |
| 3 | 7.5 |
| 11 | 7.3 |
| 20 | 6.1 |

1. A compound of formula 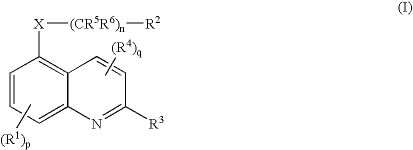
or a pharmaceutically acceptable salt or solvate thereof, wherein
p is 0, 1 or 2;
each R¹ independently represents halogen or C₁-C₆ alkyl optionally substituted by at least one substituent selected from hydroxyl, halogen and C₁-C₆ alkoxy;
X is C(O)NH or NHC(O);
n is 1, 2, 3, 4 or 5;
within each grouping, CR⁵R⁶, R⁵ and R⁶ each independently represent hydrogen, halogen, phenyl or C₁-C₆ alkyl, or R⁵ and R⁶ together with the carbon atom to which they are both attached form a C₃-C₈ cycloalkyl ring;
R² represents an unsaturated 4- to 10-membered ring system which may comprise at least one ring heteroatom selected from nitrogen, oxygen and sulphur, the ring system being optionally substituted with at least one substituent selected from halogen, —COOR¹³, hydroxyl, —NR¹⁴R¹⁵, —CONR¹⁶R¹⁷, —SO₂NR¹⁸R¹⁹, —NR²⁹SO₂R²¹, C₁-C₆ alkyl, C₁-C₆ alkylcarbonyl, C₁-C₆ alkoxy, C₁-C₆ alkylcarbonyloxy, C₁-C₆ alkoxycarbonyl, C₁-C₆ hydroxyalkyl and —S(O)ₘC₁-C₆ alkyl where m is 0, 1 or 2;
R³ represents hydrogen or a group —R⁷, —OR⁷, —SR⁷ or —NR⁷R⁸;
q is 0, 1 or 2;
each R⁴ independently represents halogen or C₁-C₆ alkyl optionally substituted by at least one substituent selected from hydroxyl, halogen and C₁-C₆ alkoxy;
R⁷ and R⁸ each independently represent hydrogen, C₁-C₁₀ alkyl, C₃-C₈ cycloalkyl or a saturated or unsaturated 3- to 10-membered heterocyclic ring system comprising at least one ring heteroatom selected from nitrogen, oxygen and sulphur, the alkyl, cycloalkyl and heterocyclic ring system each being optionally substituted with at least one substituent selected from halogen, hydroxyl, C₁-C₆ alkoxy, C₁-C₆ alkylthio, C₁-C₆ hydroxyalkyl, C₁-C₆ hydroxyalkoxy, C₁-C₆ alkoxycarbonyl, C₃-C₈ cycloalkyl, —NR⁹R¹⁰, —COOR²², —CONR²³R²⁴, —SO₂NR²⁵R²⁶, —NR²⁷SO₂R²⁸ and ZR⁶⁸ or
alternatively, R⁷ and R⁸ may together with the nitrogen atom to which they are attached form a 4- to 7-membered saturated heterocyclic ring that optionally further comprises one or two ring heteroatoms independently selected from nitrogen, oxygen and sulphur and that optionally further comprises a bridging group, the heterocyclic ring being optionally substituted with at least one substituent selected from halogen, hydroxyl, cyano, C₁-C₆ alkyl, C₁-C₆ alkoxy, C₁-C₆ alkylthio, C₁-C₆ hydroxyalkyl, C₁-C₆ hydroxyalkoxy, C₁-C₆ alkoxycarbonyl, C₃-C₈ cycloalkyl, —NR¹¹R¹², —COOR²⁹, —CONR³⁰R³¹, —SO₂NR³²R³³, —NR³⁴SO₂R³⁵, Z′R⁶⁹, (CH₂)₁₋₆NR⁷⁰R⁷¹, SO₂R⁷², NR⁷³CONR⁷⁴SO₂R⁷⁵ or M(CH₂)₁₋₆COOR⁷⁶ wherein M represents a bond, O, S, SO, SO₂, and a group >NR⁷⁷;
R⁹ and R¹⁰ each independently represent hydrogen or a C₁-C₆ alkylcarbonyl, C₂-C₇ alkenyl or C₁-C₇ alkyl group, each group being optionally substituted with at least one substituent selected from hydroxyl, —NR³⁶R³⁷, —COOR³⁸, —CONR³⁹R⁴⁰, —SO₂NR⁴¹R⁴², —NR⁴³SO₂R⁴⁴, C₁-C₆ alkoxy, C₁-C₆ alkylthio, C₁-C₆ alkoxycarbonyl and a saturated or unsaturated 3- to 10-membered ring system which may comprise at least one ring heteroatom selected from nitrogen, oxygen and sulphur, the ring system in turn being optionally substituted with at least one substituent selected from halogen, hydroxyl, oxo, carboxyl, cyano, C₁-C₆ alkyl and C₁-C₆ hydroxyalkyl, or
alternatively, R⁹ and R¹⁰ may together with the nitrogen atom to which they are attached form a 4- to 7-membered saturated heterocyclic ring that optionally further comprises one or two ring heteroatoms independently selected from nitrogen, oxygen and sulphur, the heterocyclic ring being optionally substituted with at least one substituent selected from —OR⁵⁴, —NR⁵⁵R⁵⁶, —(CH₂)ₜ—NR⁵⁷R⁵⁸ where t is 1, 2, 3, 4, 5 or 6, —COOR⁵⁹, —CONR⁶⁰R⁶¹, —SO₂NR⁶²R⁶³, —NR⁶⁴SO₂R⁶⁵, C₁-C₆ hydroxyalkyl, C₁-C₆ alkoxy, C₁-C₆ alkylthio, C₁-C₆ alkoxycarbonyl and Z″R⁸⁰;
R¹¹ and R¹² each independently represent hydrogen or a C₁-C₆ alkylcarbonyl, C₁-C₆ alkoxycarbonyl, C₂-C₇ alkenyl or C₁-C₇ alkyl group, each group being optionally substituted with at least one substituent selected from hydroxyl, —NR⁴⁵R⁴⁶, —COOR⁴⁷, —CONR⁴⁸R⁴⁹, —SO₂NR⁵⁰R⁵¹, —NR⁵²SO₂R⁵³, —NR⁶⁶C(O)R^{67,} C₁-C₆ alkoxy, C₁-C₆ alkylthio and C₁-C₆ alkoxycarbonyl;
Z, Z′ and Z″ independently represent a bond, O, S, SO, SO₂, >NR⁷⁸, C₁₋₆ alkylene, or a group —O(CH₂)₁₋₆—, —NR⁷⁹(CH₂)₁₋₆— or —S(O)ₚ(CH₂)₁₋₆— wherein p is 0, 1 or 2;
R⁶⁸, R⁶⁹ and R⁸⁰ independently represent tetrazolyl or a 5- to 6-membered heterocyclic ring comprising from 1 to 4 heteroatoms selected from nitrogen, oxygen and sulphur, which heterocyclic ring is substituted by at least one substituent selected from hydroxyl, ═O, and ═S, and which heterocyclic ring may further be optionally substituted by at least one substituent selected from halogen, nitro, cyano, —SO₂C₁₋₆ alkyl, C₁₋₆ alkoxycarbonyl, and a C₁₋₆ alkyl group which C₁₋₆ alkyl group can be optionally substituted by at least one substituent selected from halogen and hydroxyl;
R¹³, R¹⁴, R¹⁵, R¹⁶, R¹⁷, R¹⁸, R¹⁹, R²⁰ and R²¹ each independently represent hydrogen or C₁-C₆ alkyl optionally substituted by at least one substituent selected from hydroxyl, halogen and C₁-C₆ alkoxy;
R²², R²³, R²⁴, R²⁵, R²⁶, R²⁷, R²⁸, R²⁹, R³⁰, R³¹, R³², R³³, R³⁴ and R³⁵ each independently represent hydrogen or C₁-C₆ alkyl optionally substituted by at least one substituent selected from hydroxyl, halogen and C₁-C₆ alkoxy;
R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵² and R⁵³ each independently represent hydrogen or C₁-C₆ alkyl optionally substituted by at least one substituent selected from hydroxyl, halogen and C₁-C₆ alkoxy;
R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶ and R⁶⁷ each independently represent hydrogen or C₁-C₆ alkyl optionally substituted by at least one substituent selected from hydroxyl, halogen and C₁-C₆ alkoxy; and
R⁷⁰, R⁷¹, R⁷², R⁷³, R⁷⁴, R⁷⁵, R⁷⁶, R⁷⁷, R⁷⁸ and R⁷⁹ each independently represent hydrogen or C₁-C₆ alkyl optionally substituted by at least one substituent selected from hydroxyl, halogen and C 1-C₆ alkoxy;
with the provisos that:
(a) when X represents NHC(O), p is 0, q is 0, n is 1 and R³, R⁵ and R⁶ each independently represent hydrogen, then R² is other than a 2-carboxy-phenyl group; and
(b) when X represents NHC(O), p is 0, q is 0, n is 2, R³ represents hydrogen and each R⁵ and R⁶ independently represents hydrogen, then R² is other than a 3,4-diamino-phenyl group or a 5-methyl-2-furanyl group; and
(c) when X represents C(O)NH, p is 0, q is 0, n is 2, R³ represents hydrogen and each R⁵ and R⁶ independently represents hydrogen, then R² is other than an unsubstituted phenyl group, an unsubstituted 1H-indol-3-yl group, or a 2-methyl-1H-indol-3-yl group.

2. A compound according to claim 1, wherein X is NHC(O).

3. A compound according to claim 1, wherein R² represents an unsaturated 4-, 5- or 6-membered ring optionally comprising one ring heteroatom selected from nitrogen, oxygen and sulphur, the ring being optionally substituted with one, two, three or four substituents independently selected from halogen, —COOR¹³, hydroxyl, —NR¹⁴R¹⁵, —CONR¹⁶R¹⁷, —SO₂NR¹⁸R¹⁹, —NR²⁰SO₂R²¹, C₁-C₄ alkyl, C₁-C₄ alkylcarbonyl, C₁-C₄ alkoxy, C₁-C₄ alkylcarbonyloxy, C₁-C₄ alkoxycarbonyl, C₁-C₄ hydroxyalkyl and —S(O)ₘC₁-C₄ alkyl where m is 0, 1 or 2.

4. A compound according to claim 1, wherein R³ represents hydrogen or a group —R⁷ or —NR⁷R⁸.

5. A compound according to claim 1 wherein R⁷ and R⁸ each independently represent hydrogen or C₁-C₁₀ alkyl optionally substituted with one or two substituents independently selected from halogen, hydroxyl, C₁-C₄ alkoxy, C₁-C₄ alkylthio, C₁-C₄ hydroxyalkyl, C₁-C₄ hydroxyalkoxy, C₁-C₄ alkoxycarbonyl, C₅-C₆ cycloalkyl, —NR⁹R¹⁰, —COOR²², —CONR²³R²⁴, —SO₂NR²⁵R²⁶ and —NR²⁷SO₂R²⁸.

6. A compound according to claim 1, wherein R⁷ and R⁸ together with the nitrogen atom to which they are attached form a 5- to 6-membered saturated heterocyclic ring that optionally further comprises a ring nitrogen atom, the heterocyclic ring being optionally substituted with one or two substituents independently selected from halogen, hydroxyl, C₁-C₄ alkoxy, C₁-C₄ alkylthio, C₁-C₄ hydroxyalkyl, C₁-C₄ hydroxyalkoxy, C₁-C₄ alkoxycarbonyl, C₅-C₆ cycloalkyl, —NR¹¹R¹², —COOR²⁹, —CONR³⁰R³¹, —SO₂NR³²R³³ and —NR³⁴SO₂R³⁵.

7. A compound according to claim 1, wherein within each grouping CR⁵R⁶, R⁵ and R⁶ each independently represent hydrogen or C₁-C₄ alkyl.

8. A compound according to claim 1 selected from:
6-Chloro-2-methyl-N-[(2R)-2-phenylpropyl]-5-quinolinecarboxamide,
6-Chloro-2-methyl-N-[(2S)-2-phenylpropyl]-5-quinolinecarboxamide,
(βR)-N-[6-Chloro-2-[methyl[3-(methylamino)propyl]amino]-5-quinolinyl]-β-methyl-benzenepropanamide,
(βR)-N-[6-Chloro-2-(1-piperazinyl)-5-quinolinyl]-β-methyl-benzenepropanamide,
6-Chloro-2-methyl-N-(2-phenylethyl)-5-quinolinecarboxamide,
(βR)-N-[6-Chloro-2-[3-(ethylamino)propyl]-5-quinolinyl]-β-methyl-benzenepropanamide,
(βR)-N-[6-Chloro-2-[3-[(3-hydroxypropyl)amino]propyl]-5-quinolinyl]-β-methyl-benzenepropanamide,
3,4-Dichloro-α-methyl-N-5-quinolinyl-benzenepropanamide,
(βR)-N-[6-Chloro-2-[[2-[(2-hydroxyethyl)amino]ethyl]amino]-5-quinolinyl]-β-methyl-benzenepropanamide,
2-Chloro-N-[6-chloro-2-(1-piperazinyl)-5-quinolinyl]-benzenepropanamide,
2,4-Dichloro-N-[6-chloro-2-(1-piperazinyl)-5-quinolinyl]-benzenepropanamide,
4-Chloro-N-[6-chloro-2-(1-piperazinyl)-5-quinolinyl]-benzenepropanamide,
(βR)-N-[2-[(3S)-3-Amino-1-pyrrolidinyl]-6-chloro-5-quinolinyl]-β-methyl-benzenepropanamide,
N-[6-Chloro-2-(1-piperazinyl)-5-quinolinyl]-2-methoxy-benzenepropanamide,
(βR)-N-[6-Chloro-2-[(3S)-3-[(3-hydroxypropyl)amino]-1-pyrrolidinyl]-5-quinolinyl]-β-methyl-benzenepropanamide,
(βR)-N-[6-Chloro-2-[(3S)-3-[(2-hydroxyethyl)amino]-1-pyrrolidinyl]-5-quinolinyl]-β-methyl-benzenepropanamide,
N-[6-Chloro-2-(1-piperazinyl)-5-quinolinyl]-benzenepropanamide,
N-[2-[(3S)-3-Amino-1-pyrrolidinyl]-6-chloro-5-quinolinyl]-2-chlorobenzenepropanamide,
2-Chloro-N-[6-chloro-2-[(3S)-3-[(2-hydroxyethyl)amino]-1-pyrrolidinyl]-5-quinolinyl]benzenepropanamide,
1-[6-Chloro-5-[[3-(2-chlorophenyl)-1-oxopropyl]amino]-2-quinolinyl]-4-piperidinecarboxylic acid,
2-[(3S)-3-Amino-1-pyrrolidinyl]-6-chloro-N-[2-(2-chlorophenyl)ethyl]-5-quinolinecarboxamide,
6-Chloro-N-[2-(2-chlorophenyl)ethyl]-2-[(3S)-3-[(2-hydroxyethyl)amino]-1-pyrrolidinyl]-5-quinolinecarboxamide,
1-[6-Chloro-5-[[[2-(2,6-dichlorophenyl)ethyl]amino]carbonyl]-2-quinolinyl]-4-piperidinecarboxylic acid,
1-[6-Chloro-5-[[[2-(2-chlorophenyl)ethyl]amino]carbonyl]-2-quinolinyl]-4-piperidinecarboxylic acid,
1-[6-Chloro-5-[[(2,2-diphenylethyl)amino]carbonyl]-2-quinolinyl]-4-piperidinecarboxylic acid,
1-[6-Chloro-5-[[(2-phenylethyl)amino]carbonyl]-2-quinolinyl]-4-piperidinecarboxylic acid,
1-[6-Chloro-5-[[[2-(2-fluorophenyl)ethyl]amino]carbonyl]-2-quinolinyl]-4-piperidinecarboxylic acid,
1-[6-Chloro-5-[[[2-(2-methylphenyl)ethyl]amino]carbonyl]-2-quinolinyl]-4-piperidinecarboxylic acid,
1-[6-Chloro-5-[[[(2S)-2-phenylpropyl]amino]carbonyl]-2-quinolinyl]-4-piperidinecarboxylic acid,
6-Chloro-N-[2-(2-chlorophenyl)ethyl]-2-[4-(1,5-dihydro-5-oxo-4H-1,2,4-triazol-4-yl)-1-piperidinyl]-5-quinolinecarboxamide, and
1-[6-Chloro-5-[[[2-(4-chlorophenyl)ethyl]amino]carbonyl]-2-quinolinyl]-4-piperidinecarboxylic acid,
and all their pharmaceutically acceptable salts and solvates.

9. A process for the preparation of a compound of formula (I) as defined in claim 1, or a pharmaceutically acceptable salt or solvate thereof, which comprises
(a) reacting a compound of formula 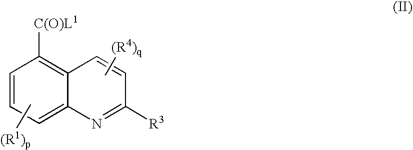
wherein L¹ represents a leaving group (e.g. hydroxyl or halogen) and p, q, R¹, R³ and R⁴ are as defined in formula (I), with a compound of formula
H₂N—(CR⁵R⁶)ₙ—R² (III)
wherein n, R², R⁵ and R⁶ are as defined in formula (I); or
(b) reacting a compound of formula 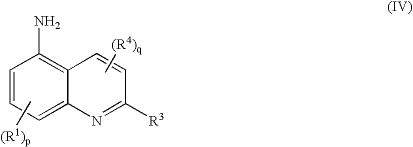
wherein p, q, R¹, R³ and R⁴ are as defined in formula (I), with a compound of formula
L²C(O)—(CR⁵R⁶)ₙ—R² (V)
wherein L² represents a leaving group (e.g. hydroxyl or halogen) and n, R², R⁵ and R⁶ are as defined in formula (I); or
(c) when R³ represents a group —NR⁷R⁸, reacting a compound of formula 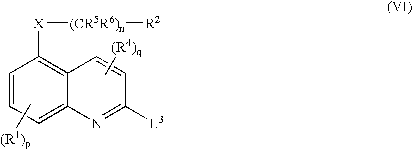
wherein L³ is a leaving group (e.g. chloride, bromide, fluoride, iodide, paratoluenesulphonate or methanesulphonate) and n, p, q, X, R¹, R², R⁴, R⁵ and R⁶ are as defined in formula (I), with a compound of formula (VII), H—NR⁷R⁸, wherein R⁷ and R⁸ are as defined in formula (I); or
(d) when R³ represents a group R⁷ where R⁷ is an optionally substituted C₃-C₁₀ alkyl group, reacting a compound of formula (VI) as defined in (c) above with a compound of formula 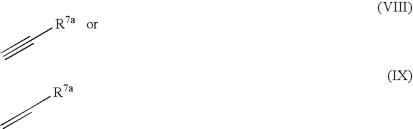
wherein R^{7a} represents a C₁-C₈ alkyl group optionally substituted as defined for R⁷ in formula (I), optionally followed by a hydrogenation reaction; or
(e) when R³ represents a group R⁷ where R⁷ is —(CH₂)₂NR⁹R¹⁰, reacting a compound of formula (VI) as defined in (c) above with a compound of formula 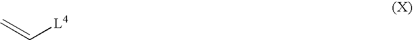
wherein L⁴ is a leaving group (eg. trialkyltin, dialkylboron or zinc), followed by reaction with a compound of formula (XI), HNR⁹R¹⁰, wherein R⁹ and R¹⁰ are as defined in formula (I); or
(f) when R³ represents a group R⁷ where R⁷ is —CH2NR⁹R¹⁰, reacting a compound of formula (VI) as defined in (c) above with a compound of formula (X) as defined in (e) above, followed by an oxidation reaction and then by reaction with a compound of formula (XI) as defined in (e) above under reductive amination conditions; or
(g) when R³ represents a group R⁷ZR⁶⁸ or NR⁷R⁸ wherein R⁷ and/or R⁸ are substituted by a group Z′R⁶⁹ or R⁷ and R⁸ together with the nitrogen atom to which they are attached form a 4- to 7-membered heterocyclic ring substituted by a group Z′R⁶⁹, and R⁶⁸ or R⁶⁹ is tetrazolyl, reacting a group of formula (XII) or (XIII) 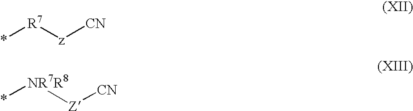
with a compound of formula GN₃, wherein G is sodium, a trialkylsilyl, an alkyltin or ammonium, to yield a group of formula I wherein R⁷, R⁸, Z, Z′ are as defined in formula (I); or
(h) when R³ represents a group R⁷ZR⁶⁸ or NR⁷R⁸ wherein R⁷ and/or R⁸ are substituted by a group Z′R⁶⁹ or R⁷ and R⁸ together with the nitrogen atom to which they are attached form a 4- to 7-membered heterocyclic ring substituted by a group Z′R⁶⁹, and R⁶⁸ or R⁶⁹ is group of formula 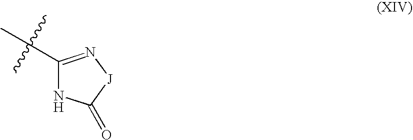
reacting a compound of formula XII or XIII wherein XII or XIII are as defined in (g) above with hydroxylamine, followed by treatment with 1,1′-thiocarbonyldiimidazole and subsequent treatment with silica gives a group of formula (XIV) wherein J is S, alternatively reacting a compound of formula XII or XIII wherein XIII or XIII are as defined in (g) above with hydroxylamine, followed by treatment with a suitable chloroformate gives a group of formula (XIV) wherein J is O; or
(i) when R³ represents a group R⁷ZR⁶⁸ or NR⁷R⁸ wherein R⁷ and/or R⁸ are substituted by a group Z′R⁶⁹ or R⁷ and R⁸ together with the nitrogen atom to which they are attached form a 4- to 7-membered heterocyclic ring substituted by a group Z′R⁶⁹, and R⁶⁸ or R⁶⁹ is 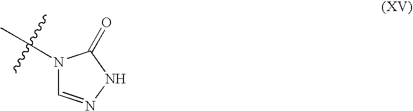
reacting a compound of formula XVI or XVII 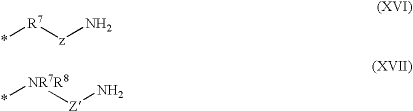
with a source of phosgene followed by treatment with formyl hydrazine and subsequent treatment with base;
and optionally after (a), (b), (c), (d), (e), (f), (g), (h) or (i) carrying out one or more of the following:
converting the compound obtained to a further compound of the invention
forming a pharmaceutically acceptable salt or solvate of the compound.

10. A compound of formula (VI) as defined in claim 9.

11. (βR)-N-(2,6-Dichloro-5-quinolinyl)-β-methyl-benzenepropanamide.

12. A pharmaceutical composition comprising a compound of formula (I) or a pharmaceutically acceptable salt or solvate thereof as claimed in claim 1 in association with a pharmaceutically acceptable adjuvant, diluent or carrier.

13. A process for the preparation of a pharmaceutical composition as claimed in claim 12 which comprises mixing a compound of formula (I) or a pharmaceutically acceptable salt or solvate thereof as defined in claim 1 with a pharmaceutically acceptable adjuvant, diluent or carrier.

14. (canceled)

15. A method of treating rheumatoid arthritis, the method comprising administering to a patient a therapeutically effective amount of a compound of formula (I) or a pharmaceutically acceptable salt or solvate thereof as claimed in claim 1.

16-17. (canceled)

18. A method of treating osteoarthritis, the method comprising administering to a patient a therapeutically effective amount of a compound of formula (I) or a pharmaceutically acceptable salt or solvate thereof as claimed in claim 1.

19. A method of treating atherosclerosis, the method comprising administering to a patient a therapeutically effective amount of a compound of formula (I) or a pharmaceutically acceptable salt or solvate thereof as claimed in claim 1.

20. A method of treating rheumatoid arthritis or osteoarthritis which comprises administering to a patient a therapeutically effective amount of a compound of formula (I) or a pharmaceutically acceptable salt or solvate thereof as claimed in claim 1.

21. A method of treating an obstructive airways disease which comprises administering to a patient a therapeutically effective amount of a compound of formula (I) or a pharmaceutically acceptable salt or solvate thereof as claimed in claim 1.

22. The method of claim 21, wherein the obstructive airways disease is asthma or chronic obstructive pulmonary disease.
